SEQUENCE LISTING (2 sequences)

SEQ ID NO: 1 - RNA, 21 nt, Artificial Sequence
  Description of Artificial Sequence Synthetic oligonucleotide
aaggcaagcugacccugaagu

SEQ ID NO: 2 - RNA, 22 nt, Artificial Sequence
  Description of Artificial Sequence Synthetic oligonucleotide
ucaacaucagucugauaagcua

(19) United States
(12) Patent Application Publication
(10) Pub. No.: US 2012/0027753 A1
(43) Pub. Date: Feb. 2, 2012

(54) MicroRNAs in Never-Smokers and Related Materials and Methods

(75) Inventors: Carlo M. Croce, Columbus, OH (US); Curtis C. Harris, Garrett Park, MD (US); Masahiro Seike, Tokyo (JP); Izumi Horikawa, Rockville, MD (US)

(73) Assignees: THE OHIO STATE UNIVERSITY, Columbus, OH (US); Department of Health and Human Sevices, Washington, DC (US); The Government of the United States of America as Represented by the Secretary of the

(21) Appl. No.: 13/202,666

(22) PCT Filed: Feb. 24, 2010
(86) PCT No.: PCT/US10/25173

§ 371 Date: Sep. 26, 2011

Related U.S. Application Data
(60) Provisional application No. 61/155,709, filed on Feb. 26, 2009.

(51) Int. Cl. A61K 31/7088; A61P 35/00; C12N 5/02; C07H 21/02; C07K 16/00; C12Q 1/68; A61K 39/395; A61P 35/02

(52) U.S. Cl. 424/133.1; 514/44 A; 424/142.1; 536/24.5; 530/387.1; 435/6.14; 435/375; 435/6.13

(57) ABSTRACT

The present invention provides novel methods and compositions for the diagnosis, prognosis and treatment of lung cancer in never-smokers. The invention also provides methods of identifying anti-lung cancer agents.

### CROSS-REFERENCE TO RELATED APPLICATIONS

This application claims the benefit of U.S. Provisional Application No. 61/155,709, filed Feb. 26, 2009, the disclosure of which is incorporated herein by reference.

### STATEMENT REGARDING FEDERALLY SPONSORED RESEARCH

This invention was made with government support under the Intramural Research Program and the National Institutes of Health, National Cancer Institute, and Center for Cancer Research. The government has certain rights in the invention.

### SEQUENCE LISTING

The instant application contains a Sequence Listing which has been submitted via EFS-web and is hereby incorporated by reference in its entirety. The ASCII copy, created on Feb. 23, 2010, is named 604_{—}50753_SEQ_LIST_{—}06008-2.txt, and is 758 bytes in size.

### BACKGROUND OF THE INVENTION

Lung cancer is the leading cause of cancer death and the most common cause of smoking-related mortality both in the United States and worldwide. However, approximately 10-25% of all lung cancer cases are not attributable to smoking. Recent studies that pay specific attention to lung cancers in never-smokers have suggested that they have distinct characteristics from those in smokers: G to T transversions of the p53 and K-ras mutations occur less frequently in lung adenocarcinomas from never-smokers than in those from smokers; and mutations of epidermal growth factor receptor (EGFR) gene are more frequently observed in never-smoker cases.

EGFR tyrosine kinase inhibitors (EGFR-TKIs), including gefitinib and erlotinib, are currently in clinical use and preferentially effective in EGFR mutant cases. However, as much as 30% of EGFR mutant cases and 90% of EGFR wild-type cases showed no therapeutic response to EGFR-TKIs.

MicroRNAs [interchangeably: “MiRNAs,” “miR(s),” which is (are) the gene product(s) of “miR(s)”] are small non-coding RNA molecules of about 18-25 nucleotides encoded by genes that are frequently located at chromosomal regions deleted or amplified in cancers, suggesting that miRs are a new class of genes involved in human tumorigenesis. Expression levels of miRs are altered in various types of human cancers, including lung cancers. Recently, miRs have been demonstrated as diagnostic and prognostic markers in leukemia, lung cancer and colon cancer. The inventors herein now believe that miRs can be a therapeutic target in human cancers.

The inventors herein have previously analyzed miR expression profiles of 104 lung cancers, 99 of which were from smokers, and found that high miR-155 and low let-7a correlated with poor survival.

In short, identification of new therapeutic targets and methods to improve EGFR-TKI therapy is of critical importance to the better treatment of cancer, particularly lung cancer.

### SUMMARY OF THE INVENTION

The invention is based, at least in part, on the inventors' discovery of a global expression profile of miRs in lung cancers from never-smokers. Comparisons of miR expression profiles in never-smoker versus smoker cases and in EGFR wild-type versus EGFR mutant cases show a unique etiology of lung cancers from never-smokers and reveal EGFR-mediated regulation of miR expression.

The in vitro functional analyses presented herein also shows that the modulation of certain genes encoding miRs or their gene products, are therapeutic either alone, in combination with other such modulators, in combination with other cancer treatments, and can be a therapeutic in combination with EGFR-TKI treatment.

In a broad embodiment, the present invention provides compositions composition of matter comprising at least one anti-sense miR and at least one additional composition, wherein the anti-sense miR is anti-sense to a miR that is differentially expressed in epidermal growth factor receptor never-smoker mutant cancer cells compared to wild-type never-smoker cancer cells, and wherein the at least one additional composition is useful to treat cancer. In certain embodiments, the at least one additional composition can be selected from the group comprising: a chemotherapy drug; AG1478; gefitinib (Iressa®); erlotinib (Tarceva®); cetuximab; panitumab; zalutumamab; nimotuzamab; matuzumab; and lapatinib.

Also, in certain embodiments, such compositions where the anti-sense miR is selected from a miR that is anti-sense to a miR can be selected from the group: miR-21; miR-210; miR-129. Also, in certain embodiments, such compositions can include wherein the at least one anti-sense miR is anti-sense to miR-21; those wherein the at last one additional composition useful to treat cancer is an epidermal growth factor receptor tyrosine kinase inhibitor; or preferably wherein the epidermal growth factor receptor tyrosine kinase inhibitor is AG1478.

Also provided by the present invention are compositions of matter comprising at least one anti-sense miR and at least one additional composition, wherein the miR is upregulated in epidermal growth factor receptor mutant never-smoker cancer cells compared to wild-type never-smoker cancer cells, and wherein the at least one additional composition is useful to treat cancer. In certain embodiments, the miR is selected from the group: miR-486; miR-126; miR-138; miR-521; miR-451; miR-141; miR-30d; and miR-30a.

Also provided are compositions of matter comprising at least one anti-sense miR and at least one composition, wherein the anti-sense miR is anti-sense to a miR that is upregulated in EGFR mutant never-smoker cancer cells compared to wild-type never-smoker cancer cells, and wherein the at least one additional composition is useful to treat cancer. In certain embodiments, those compositions wherein the anti-sense miR can be selected from a miR that is anti-sense to a miR selected from the group: miR-21; miR-210; and miR-129.

In other broad embodiments, there are provided methods to identify epidermal growth factor receptor mutant cancer cells in a test sample, comprising comparing miR levels in a test sample to miR levels of a control, wherein differentially-expressed miR levels identify the test sample as containing epidermal growth factor receptor mutant cancer cells. In certain embodiments, those methods include wherein the miR are selected from the group comprising: miR-21; miR-210; miR-129; miR-486; miR-126; miR-138; miR-521; miR-451; miR-141; miR-30d; and miR-30a.

In other broad embodiments, there are provided methods to determine whether a never-smoker subject has, or is at risk for developing, lung cancer, comprising comparing miR levels in a test sample to miR levels of a control, wherein differentially-expressed miR levels diagnoses the subject as either having, or being at risk for developing, lung cancer. In certain embodiments, such methods can further comprise comparing epidermal growth factor receptor mutant status in the test sample and control. Also, in certain embodiments, those methods can include wherein the epidermal growth factor receptor mutant status is determined using an epidermal growth factor receptor tyrosine kinase inhibitor. Also preferred are those methods as described, wherein the miR is selected from the group: miR-21; miR-210; miR-129; miR-486; miR-126; miR-138; miR-521; miR-451; miR-141; miR-30d; and miR-30a.

In other broad embodiments, there are provided methods to provide a prognosis in a never-smoker cancer patient, comprising: comprising comparing miR levels in a test sample to miR levels of a control, wherein differentially-expressed miR levels indicates a poor prognosis. In certain embodiments, those methods can include wherein the miR is selected from the group: miR-21; miR-210; miR-129; miR-486; miR-126; miR-138; miR-521; miR-451; miR-141; miR-30d; and miR-30a.

In another broad embodiment, there are provided methods of diagnosing epidermal growth factor receptor-mutant cancer in a patient, comprising comparing miR levels in a test sample to miR levels of a control, wherein differentially-expressed miR levels diagnoses the subject as having epidermal growth factor receptor-mutant cancer. In certain embodiments, methods can include wherein the miR is selected from the group: miR-21; miR-210; miR-129; miR-486; miR-126; miR-138; miR-521; miR-451; miR-141; miR-30d; and miR-30a.

In another broad embodiment, there are provided methods to provide a prognosis in epidermal growth factor receptor-mutant cancer patient, comprising: comprising comparing miR levels in a test sample to miR levels of a control, wherein differentially-expressed miR levels indicates a poor prognosis. In certain embodiments, those methods can include wherein the miR is selected from the group: miR-21; miR-210; miR-129; miR-486; miR-126; miR-138; miR-521; miR-451; miR-141; miR-30d; and miR-30a.

In another broad embodiment, there are provided methods to treat cancer in a never-smoker patient in need of such treatment, comprising administering a pharmaceutically-effective amount of a composition herein. In certain embodiments, the methods include wherein the cancer treated is selected from the group comprising: neuroblastoma; lung cancer; bile duct cancer; non small cell lung carcinoma; hepatocellular carcinoma; lymphoma; nasopharyngeal carcinoma; ovarian cancer; head and neck squamous cell carcinoma; squamous cell cervical carcinoma; gastric cancer; colon cancer; uterine cervical carcinoma; gall bladder cancer; prostate cancer; breast cancer; testicular germ cell tumors; large cell lymphoma; follicular lymphoma; colorectal cancer; malignant pleural mesothelioma; glioma; thyroid cancer; basal cell carcinoma; T cell lymphoma; t(8;17)-prolyphocytic leukemia; myelodysplastic syndrome; pancreatic cancer; t(5;14)(q35.1;q32.2) leukemia; malignant fibrous histiocytoma; gastrointestinal stromal tumor; and hepatoblastoma.

In another broad embodiment, there are provided methods to treat cancer in a never-smoker patient in need of such treatment, comprising administering a pharmaceutically-effective amount of a composition that is anti-sense to miR-21. In certain embodiments, those methods can include wherein the cancer treated is a lung cancer. Also, in a particular embodiment, those methods can further comprise administering anti-sense miR-21 and an epidermal growth factor receptor tyrosine kinase inhibitor; and in certain embodiments, wherein the epidermal growth factor receptor tyrosine kinase inhibitor is AG1478. In one particular embodiment, those methods include wherein the cancer treated is adenocarcinoma.

In another broad embodiment, there are provided methods to treat cancer in a never-smoker patient in need of such treatment, comprising administering a pharmaceutically-effective amount of an anti-sense miR, wherein the antisense miR is antisense to a miR selected from the group: miR-21; miR -210; miR-129.

In another broad embodiment, there are provided methods to treat cancer in a never-smoker patient in need of such treatment, comprising administering a pharmaceutically-effective amount of an anti-sense miR, wherein the antisense miR is antisense to miR-21. In certain embodiments, those methods can include wherein the cancer treated is selected from the group comprising: neuroblastoma and lung cancer. Also, in certain embodiments, those methods can include wherein the cancer treated is adenocarcinoma. Also, in certain embodiments, those methods can further comprise administering an adjuvant. Also, in certain embodiments, those methods can further comprise administering a compound selected from the group: compound selected from the group comprising: a chemotherapy drug; AG1478; gefitinib (Iressa®); erlotinib (Tarceva®); cetuximab; panitumab; zalutumamab; nimotuzamab; matuzumab; and lapatinib. Also, in certain embodiments, those methods can further comprise administering an epidermal growth factor receptor tyrosine kinase inhibitor. Also, in certain embodiments, those methods can further comprise administering AG1478, or a pharmaceutically-acceptable formulation thereof.

In another broad embodiment, there are provided methods to treat an epidermal growth factor receptor mutant cancer in a patient in need of such treatment, comprising administering a pharmaceutically-effective amount of a composition herein. Also provided are to treat an epidermal growth factor receptor mutant cancer in a patient in need of such treatment, comprising administering a pharmaceutically-effective amount of an anti-sense miR-21.

In another broad embodiment, there are provided methods to treat an epidermal growth factor receptor mutant cancer in a patient in need of such treatment, comprising administering a pharmaceutically-effective amount of a miR expression inhibitor, wherein the miR is selected from the group: miR-21; miR-210; and miR-129. Also provided are methods to treat an epidermal growth factor receptor mutant cancer in a patient in need of such treatment, comprising administering a pharmaceutically-effective amount of a miR-21 expression inhibitor. In certain embodiments, those methods can further comprise administering a compound selected from the group comprising: a chemotherapy drug; AG1478; gefitinib (Iressa®); erlotinib (Tarceva®); cetuximab; panitumab; zalutumamab; nimotuzamab; matuzumab; and lapatinib. Also, in certain embodiments, those methods can further comprise administering an epidermal growth factor receptor tyrosine kinase inhibitor. Also, in certain embodiments, those methods can further comprise administering AG1478, or a pharmaceutically-acceptable formulation thereof.

In another broad embodiment, there are provided methods to treat an epidermal growth factor receptor mutant cancer in a patient in need of such treatment, comprising administering a pharmaceutically-effective amount of a miR expression promoting composition, wherein the miR is selected from the group: miR-486; miR-126; miR-138; miR-521; miR-451; miR-141; miR-30d; and miR-30a.

In another broad embodiment, there are provided methods for inducing apoptosis of epidermal growth factor receptor mutant cancer cells, comprising introducing an apoptosis-effective amount of a composition herein.

In another broad embodiment, there are provided methods for inducing apoptosis of epidermal growth factor receptor mutant cancer cells, comprising introducing an apoptosis-effective amount of a composition comprising an anti-sense miR-21 in combination with a, epidermal growth factor receptor tyrosine (EGFR) kinase inhibitor.

In another broad embodiment, there are provided methods for inducing apoptosis of epidermal growth factor receptor mutant cancer cells, comprising introducing an apoptosis-effective amount of an anti-sense miR, wherein the antisense miR is antisense to miR-21. In certain embodiments, those methods can include wherein the epidermal growth factor receptor mutant cancer cells are adenocarcinoma cells. Also, in certain embodiments, those methods can include wherein the adenocarcinoma cells are selected from the group: H3255 cells; H1975 cells; and H1650 cells. Also, in certain embodiments, those methods can further-comprise introducing an adjuvant. Also, in certain embodiments, those methods can further comprise introducing a composition selected from the group: a chemotherapy drug; a stem cell; AG1478; gefitinib (Iressa®); erlotinib (Tarceva®); cetuximab; panitumab; zalutumamab; nimotuzamab; matuzumab; and lapatinib. Also, in certain embodiments, those methods can further comprise administering an epidermal growth factor receptor tyrosine kinase inhibitor. Also, in certain embodiments, those methods can further comprise administering AG1478, or a pharmaceutically-acceptable formulation thereof.

In another broad embodiment, there are provided methods for inducing apoptosis of epidermal growth factor receptor mutant cancer cells, comprising introducing an apoptosis-effective amount of a miR expression inhibitor, wherein the miR is selected from the group comprising: miR-21; miR-210; and miR-129.

In another broad embodiment, there are provided methods for inducing apoptosis of epidermal growth factor receptor mutant cancer cells, comprising introducing an apoptosis-effective amount of a miR-21 expression inhibitor. In certain embodiments, those methods can further comprises administering a compound selected from the group comprising: a chemotherapy drug; a stem cell; AG1478; gefitinib (Iressa®); erlotinib (Tarceva®); cetuximab; panitumab; zalutumamab; nimotuzamab; matuzumab; and lapatinib. Also, in certain embodiments, those methods can further comprise administering an epidermal growth factor receptor tyrosine kinase inhibitor. Also, in certain embodiments, those methods can further comprise administering AG1478, or a pharmaceutically-acceptable formulation thereof.

In another broad embodiment, there are provided methods for inducing apoptosis of epidermal growth factor receptor mutant cancer cells, comprising introducing an apoptosis-effective amount of a miR expression promoting composition, wherein the miR is selected from the group: miR-486; miR-126; miR-138; miR-521; miR-451; miR-141; miR-30d; and miR-30a.

In another broad embodiment, there are provided methods for identifying pharmaceutically-useful compositions, comprising: i) introducing an anti-sense miR to an epidermal growth factor receptor mutant cancer cell culture, wherein the anti-sense miR is anti-sense to a miR selected from the group of: miR-21; miR-210; miR-129; miR-486; miR-126; miR-138; miR-521; miR-451; miR-141; miR-30d; and miR-30a; ii) introducing a test composition to the epidermal growth factor receptor mutant cancer cell culture; and, iii) identifying test compositions which induce apoptosis as pharmaceutically-useful compositions.

In another broad embodiment, there are provided methods for identifying pharmaceutically-useful compositions, comprising: i) introducing an anti-sense miR to an epidermal growth factor receptor mutant cancer cell culture, wherein the anti-sense miR is anti-sense to miR-21; ii) introducing a test composition to the epidermal growth factor receptor mutant cancer cell culture; and iii) identifying test compositions which induce apoptosis as pharmaceutically-useful compositions. In certain embodiments, those methods can include wherein the cancer cells are a lung cancer cells. Also, in certain embodiments, those methods can further comprise a step of identifying phosphorylated epidermal growth factor receptor levels.

In another broad embodiment, there are provided methods of predicting the clinical outcome of a patient diagnosed with lung cancer, comprising detecting the expression level of miR-21 in a cancer cell sample obtained from the patient, wherein a 1.5-fold or greater increase in the level of miR-21 relative to a control, in combination with a epidermal growth factor receptor mutant status predicts a decrease in survival.

In another broad embodiment, there are provided methods to identify a therapeutic agent for the treatment of lung cancer, comprising screening candidate agents in vitro to select an agent that decreases expression of miR-21, thereby identifying an agent for the treatment of lung cancer.

In another broad embodiment, there are provided kits for identifying a differentially-expressed miR in lung cancer, comprising at least one molecular identifier of a miR selected from the group: miR-21; miR-210; miR-129; miR-486; miR-126; miR-138; miR-521; miR-451; miR-141; miR-30d; and miR-30a.

In another broad embodiment, there are provided kits for identifying a differentially-expressed miR-21 in lung cancer, comprising at least one molecular identifier of miR-21, wherein the molecular identifier is selected from the group: probes; primers; antibodies; miR; locked miR; or small molecule.

Various objects and advantages of this invention will become apparent to those skilled in the art from the following detailed description of the preferred embodiment, when read in light of the accompanying drawings.

### BRIEF DESCRIPTION OF THE DRAWINGS

FIGS. 1A-1B: MiR-21 expression in human lung cancer cell lines.

FIG. 1A: MiR-21 expression levels were analyzed by qRT-PCR and expressed relative to HBET2 (hTERT-immortalized normal human bronchial epithelial cells) (defined as 1.0, not shown). Data were mean±SD from three independent experiments. *, p<0.05 when compared with HBET2, Student t-test. The suppressive effects of AG1478 on cell growth were determined by MTS assay and indicated as IC50 (half maximal inhibitory concentration). Sq, squamous cell carcinoma; La, large cell carcinoma; Ad, adenocarcinoma; S, derived from smoker cases; N, derived from never-smoker cases; N/A, information not available; Wt, EGFR wild-type; Mt*, EGFR mutant ΔE746-A750; Mt**, L858R and T790M; Mt***, L858R.

FIG. 1B: Correlation between miR-21 expression and p-EGFR levels (Pearson's correlation, r=0.71, p<0.05). The miR-21 data were from (FIG. 1A) and the p-EGFR data were obtained by quantitatively analyzing the results shown in FIG. 6.

FIGS. 2A-2B: AG1478 represses miR-21 expression. H3255 lung adenocarcinoma cells, characterized by a high expression of miR-21 and EGFR mutation, were serum-starved for 24 h and then grown in either the presence or absence of AG1478 (2 μM or 10 μM) for 2 h with or without following exposure to 20 ng/ml of EGF for 15 min.

FIG. 2A: The effect of AG1478 on phospho-EGFR (p-EGFR) and phospho-Akt (p-Akt) expression. β-actin was a loading control.

FIG. 2B: MiR-21 expression levels analyzed by qRT-PCR after the AG1478 treatments (2 μM or 10 μM) with or without EGF ligand stimulation. MiR-21 expression levels were expressed as the relative values to untreated cells in the absence of EGF. Data were mean±SD from four independent experiments. *, p<0.05, paired t-test.

FIGS. 3A-3D: Inhibition of miR-21 enhances AG1478-induced apoptosis.

FIG. 3A: Cells were transfected with 40 nM of anti-miR-21 (+) or control oligonucleotide (anti-EGFP) (−) for 72 h and examined by qRT-PCR. The expression levels of miR-21 after transfection of anti-miR-21 were expressed as the relative values to control. Data were mean±SD from three independent experiments. *, p<0.05, paired t-test.

FIGS. 3B-3C: Cells (H3255 or H441) were transfected with 40 nM of anti-miR-21 (+) or anti-EGFP (−) for 72 h and then grown in the presence or absence of 0.2 μM of AG1478 for 24 h (H3255) or 10 μM for 72 h (H441). The activities of caspase 3/7 were expressed as the relative values to the activities of cells without anti-miR-21 and AG1478. Data were mean±SD from at least four independent experiments. *, p<0.05, Student t-test.

FIG. 3D: Uncleaved PARP was evaluated by Western blot. Cells were transfected with anti-miR-21 or anti-EGFP as above and then grown in the presence or absence of 2 μM of AG1478 for 72 h. β-actin was a loading control.

FIGS. 4A-4C: MiR-21 (FIG. 4A), miR-126 (FIG. 4B) and miR-486 (FIG. 4C) expression from never-smoker samples. Expression levels of each miR in 20 pairs of tumor and normal tissues were analyzed using qRT-PCR. Fifteen cases were EGFR wild-type (case no. 1, 3, 5, 6, 8, 9, 10, 11, 12, 13, 14, 15, 23, 26 and 27), and 5 cases were EGFR mutant (case no. 2, 4, 24, 25 and 28). The five tumors expressing high levels of miR-21 were from three EGFR mutants (case no. 24, 25 and 28) and two EGFR wild-type (case no. 5 and 23) cases. Reactions were in triplicate for each sample. The expression levels were normalized with RNU6B, determined using the 2-ΔΔCT method, and presented relative to the mean value of normal tissue. *, p<0.05, paired t-test.

FIGS. 5A-5C: MiR-21 (FIG. 5A), miR-126* (FIG. 5B) and miR-138 (FIG. 5C) expression in never-smokers versus smokers. Thirteen pairs from never-smokers and 14 pairs from smokers of lung adenocarcinoma and normal lung tissues were analyzed by qRT-PCR. Reactions were in triplicate for each sample. Relative expression was quantified as Log 2 2-ΔCT, where ΔCT=(CTmiR-CTRNU6B). *, p<0.05, paired t-test.

FIG. 6: Western blot analysis of eight NSCLC cell lines. Protein expressions of phospho-EGFR (p-EGFR), EGFR and phospho-Akt (p-Akt) were examined by Western blot analysis. A; non-adenocarcinoma cell lines (squamous cell carcinoma H157 and large cell carcinoma H1299), B; adenocarcinoma cell lines with wild-type EGFR (A549, H23 and H441), C; adenocarcinoma cell lines with mutant EGFR (H1650, H1975 and H3255). β-actin was a loading control. These images were quantified by measuring signal intensity using NIH Image J1.40g.

FIG. 7: AG1478 represses miR-21 expression in H441 lung adenocarcinoma cells. MiR-21 expression levels were analyzed by qRT-PCR after the AG1478 treatments (2 μM or 10 μM) in the absence of EGF and expressed relative to untreated cells. Data were mean±SD from triplicate. *, p<0.05, paired t-test.

FIG. 8: Table 1—Characteristics of never-smoker patients with non-small cell lung cancer.

FIG. 9: Table 2—Characteristics of never-smoker patients with non-small cell lung cancer (n=28).

FIG. 10: Table 3—miRs differentially expressed between lung cancer tissues and normal lung tissues from 28 never-smokers.

FIG. 11: Table 4 Characteristics of smoker patients with lung adenocarcinoma (n=23).

FIGS. 12A-C: Table 5—Forty-three miRs differentially expressed and related to smoking status.

FIG. 13—Table 6—miRs differentially expressed between EGFR mutant and wild-type lung cancers from never-smokers.

### DETAILED DESCRIPTION OF THE INVENTION

Approximately 15% of lung cancer cases are not associated with smoking and show molecular and clinical characteristics distinct from those in smokers. Epidermal growth factor receptor (EGFR) gene mutations, which are correlated with sensitivity to EGFR-tyrosine kinase inhibitors (EGFR-TKIs), are more frequent in never-smoker lung cancers.

It is now shown herein that microRNA (miR) expression profiling of 28 never-smoker lung cancer cases identified aberrantly expressed miRs, which were much fewer than in lung cancers of smokers and included miRs previously identified (e.g., upregulated miR-21) and unidentified (e.g., downregulated miR-138) in those smoker cases.

The changes in expression of some of these miRs were more remarkable in cases with EGFR mutations than in those without: the most upregulated miR, miR-21, was more abundant in cancers with EGFR mutation. A significant correlation between phosphorylated-EGFR (p-EGFR) and miR-21 levels in lung carcinoma cell lines and the suppression of miR-21 by an EGFR-TKI, AG1478, now shows that the EGFR signaling pathway positively regulated miR-21 expression. In a never-smoker-derived lung adenocarcinoma cell line H3255 with mutant EGFR and high levels of p-EGFR and miR-21, antisense inhibition of miR-21 enhanced AG1478-induced apoptosis. In a never-smoker-derived adenocarcinoma cell line H441 with wild-type EGFR, the antisense miR-21 not only showed the additive effect with AG1478 but also induced apoptosis by itself. The aberrantly increased expression of miR-21, which is further enhanced by the activated EGFR signaling pathway, plays a role in lung carcinogenesis in never-smokers and is a potential therapeutic target in both EGFR mutant and wild-type cases.

The present invention therefore provides materials and methods related to these new discoveries. In particular, compositions useful to treat cancers, particularly lung cancers are provided. However, also provided are methods to identify additional compositions useful to treat cancers, methods to diagnose cancers, methods to provide prognosis of cancers, methods to induce apoptosis, etc. Also provided are research tools associated with these discoveries, particularly kits and the like.

Abbreviations

DNA Deoxyribonucleic acid

mRNA Messenger RNA

PCR Polymerase chain reaction

pre-miR Precursor microRNA

qRT-PCR Quantitative reverse transcriptase polymerase chain reaction

RNA Ribonucleic acid

Terms

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

It is understood that a miR is derived from genomic sequences or a gene. In this respect, the term “gene” is used for simplicity to refer to the genomic sequence encoding the precursor miR for a given miR. However, embodiments of the invention may involve genomic sequences of a miR that are involved in its expression, such as a promoter or other regulatory sequences.

The term “miR” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miR may have a self-complementary region, which is up to 100% complementary miR probes of the invention can be or be at least 60, 65, 70, 75, 80, 85, 90, 95, or 100% complementary to their target.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Control: A “control” refers to a sample or standard used for comparison with an experimental sample, such as a tumor sample obtained from a patient.

Cytokines: Proteins produced by a wide variety of hematopoietic and non-hematopoietic cells that affect the behavior of other cells. Cytokines are important for both the innate and adaptive immune responses.

Decrease in survival: As used herein, “decrease in survival” refers to a decrease in the length of time before death of a patient, or an increase in the risk of death for the patient.

Detecting level of expression: For example, “detecting the level of miR or miR expression” refers to quantifying the amount of miR or miR present in a sample. Detecting expression of the specific miR, or any microRNA, can be achieved using any method known in the art or described herein, such as by qRT-PCR. Detecting expression of miR includes detecting expression of either a mature form of miR or a precursor form that is correlated with miR expression. Typically, miR detection methods involve sequence specific detection, such as by RT-PCR. miR-specific primers and probes can be designed using the precursor and mature miR nucleic acid sequences, which are known in the art and provided herein as in the SEQ ID NOs.

MicroRNA (miR): Single-stranded RNA molecules that regulate gene expression. MicroRNAs are generally 21-23 nucleotides in length. MicroRNAs are processed from primary transcripts known as pri-miR to short stem-loop structures called precursor (pre)-miR and finally to functional, mature microRNA. Mature microRNA molecules are partially complementary to one or more messenger RNA molecules, and their primary function is to down-regulate gene expression. MicroRNAs regulate gene expression through the RNAi pathway.

miR expression: As used herein, “low miR expression” and “high miR expression” are relative terms that refer to the level of miRs found in a sample. In some embodiments, low and high miR expression is determined by comparison of miR levels in a group of control samples and test samples. Low and high expression can then be assigned to each sample based on whether the expression of mi in a sample is above (high) or below (low) the average or media miR expression level. For individual samples, high or low miR expression can be determined by comparison of the sample to a control or reference sample known to have high or low expression, or by comparison to a standard value. Low and high miR expression can include expression of either the precursor or mature forms of miR, or both.

Patient: As used herein, the term “patient” includes human and non-human animals. The preferred patient for treatment is a human. “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Screening: As used herein, “screening” refers to the process used to evaluate and identify candidate agents that affect such disease. Expression of a microRNA can be quantified using any one of a number of techniques known in the art and described herein, such as by microarray analysis or by qRT-PCR.

Small molecule: A molecule, typically with a molecular weight less than about 1000 Daltons, or in some embodiments, less than about 500 Daltons, wherein the molecule is capable of modulating, to some measurable extent, an activity of a target molecule.

Therapeutic: A generic term that includes both diagnosis and treatment.

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject.

As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically effective amount: A quantity of a specified pharmaceutical or therapeutic agent sufficient to achieve a desired effect in a subject, or in a cell, being treated with the agent. The effective amount of the agent will be dependent on several factors, including, but not limited to the subject or cells being treated, and the manner of administration of the therapeutic composition.

In some embodiments of the present methods, use of a “control” is desirable. In that regard, the control may be a non-cancerous tissue sample obtained from the same patient, or a tissue sample obtained from a healthy subject, such as a healthy tissue donor. In another example, the control is a standard calculated from historical values. Tumor samples and non-cancerous tissue samples can be obtained according to any method known in the art. For example, tumor and non-cancerous samples can be obtained from cancer patients that have undergone resection, or they can be obtained by extraction using a hypodermic needle, by microdissection, or by laser capture. Control (non-cancerous) samples can be obtained, for example, from a cadaveric donor or from a healthy donor.

In some embodiments, screening comprises contacting the candidate agents with cells. The cells can be primary cells obtained from a patient, or the cells can be immortalized or transformed cells.

The “candidate agents” can be any type of agent, such as a protein, peptide, small molecule, antibody or nucleic acid. In some embodiments, the candidate agent is a cytokine. In some embodiments, the candidate agent is a small molecule. Screening includes both high-throughout screening and screening individual or small groups of candidate agents.

In some methods herein, it is desirable to identify miRs present in a sample.

The sequences of precursor microRNAs (pre-miRs) and mature miRs are publicly available, such as through the miRBase database, available online by the Sanger Institute (see Griffiths-Jones et al., Nucleic Acids Res. 36:D154-D158, 2008; Griffiths-Jones et al., Nucleic Acids Res. 34:D140-D144, 2006; and Griffiths-Jones, Nucleic Acids Res. 32:D109-D111, 2004). The sequences of the precursor and mature forms of the presently disclosed preferred family members are provided herein.

Detection and quantification of RNA expression can be achieved by any one of a number of methods well known in the art (see, for example, U.S. Patent Application Publication Nos. 2006/0211000 and 2007/0299030, herein incorporated by reference) and described below. Using the known sequences for RNA family members, specific probes and primers can be designed for use in the detection methods described below as appropriate.

In some cases, the RNA detection method requires isolation of nucleic acid from a sample, such as a cell or tissue sample. Nucleic acids, including RNA and specifically miR, can be isolated using any suitable technique known in the art. For example, phenol-based extraction is a common method for isolation of RNA. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g., precursor and mature miRs, 5S and 5.8S ribosomal RNA (rRNA), and U1 small nuclear RNA (snRNA)). In addition, extraction procedures such as those using TRIZOL™ or TRI REAGENT™, will purify all RNAs, large and small, and are efficient methods for isolating total RNA from biological samples that contain miRs and small interfering RNAs (siRNAs).

In some embodiments, use of a microarray is desirable. A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used, for example, to measure the expression levels of large numbers of messenger RNAs (mRNAs) and/or miRs simultaneously.

Microarrays can be fabricated using a variety of technologies, including printing with fine-pointed pins onto glass slides, photolithography using pre-made masks, photolithography using dynamic micromirror devices, ink jet printing, or electrochemistry on microelectrode arrays.

Microarray analysis of miRs, for example (although these procedures can be used in modified form for any RNA analysis) can be accomplished according to any method known in the art (see, for example, PCT Publication No. WO 2008/054828; Ye et al., Nat. Med. 9(4):416-423, 2003; Calin et al., N. Engl. J. Med. 353(17):1793-1801, 2005, each of which is herein incorporated by reference). In one example, RNA is extracted from a cell or tissue sample, the small RNAs (18-26-nucleotide RNAs) are size-selected from total RNA using denaturing polyacrylamide gel electrophoresis. Oligonucleotide linkers are attached to the 5′ and 3′ ends of the small RNAs and the resulting ligation products are used as templates for an RT-PCR reaction with 10 cycles of amplification. The sense strand PCR primer has a fluorophore attached to its 5′ end, thereby fluorescently labeling the sense strand of the PCR product. The PCR product is denatured and then hybridized to the microarray. A PCR product, referred to as the target nucleic acid that is complementary to the corresponding miR capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miR, using a number of positive and negative controls and array data normalization methods, which will result in assessment of the level of expression of a particular miR.

In an alternative method, total RNA containing the small RNA fraction (including the miR) extracted from a cell or tissue sample is used directly without size-selection of small RNAs, and 3′ end labeled using T4 RNA ligase and either a fluorescently-labeled short RNA linker. The RNA samples are labeled by incubation at 30° C. for 2 hours followed by heat inactivation of the T4 RNA ligase at 80° C. for 5 minutes. The fluorophore-labeled miRs complementary to the corresponding miR capture probe sequences on the array will hybridize, via base pairing, to the spot at which the capture probes are affixed. The microarray scanning and data processing is carried out as described above.

There are several types of microarrays than be employed, including spotted oligonucleotide microarrays, pre-fabricated oligonucleotide microarrays and spotted long oligonucleotide arrays. In spotted oligonucleotide microarrays, the capture probes are oligonucleotides complementary to miR sequences. This type of array is typically hybridized with amplified PCR products of size-selected small RNAs from two samples to be compared (such as non-cancerous tissue and cancerous or sample tissue) that are labeled with two different fluorophores. Alternatively, total RNA containing the small RNA fraction (including the miRs) is extracted from the two samples and used directly without size-selection of small RNAs, and 3′ end labeled using T4 RNA ligase and short RNA linkers labeled with two different fluorophores. The samples can be mixed and hybridized to one single microarray that is then scanned, allowing the visualization of up-regulated and down-regulated miR genes in one assay.

In pre-fabricated oligonucleotide microarrays or single-channel microarrays, the probes are designed to match the sequences of known or predicted miRs. There are commercially available designs that cover complete genomes (for example, from Affymetrix or Agilent). These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing.

In some embodiments, use of quantitative RT-PCR is desirable. Quantitative RT-PCR (qRT-PCR) is a modification of polymerase chain reaction used to rapidly measure the quantity of a product of polymerase chain reaction. qRT-PCR is commonly used for the purpose of determining whether a genetic sequence, such as a miR, is present in a sample, and if it is present, the number of copies in the sample. Any method of PCR that can determine the expression of a nucleic acid molecule, including a miR, falls within the scope of the present disclosure. There are several variations of the qRT-PCR method known in the art, three of which are described below.

Methods for quantitative polymerase chain reaction include, but are not limited to, via agarose gel electrophoresis, the use of SYBR Green (a double stranded DNA dye), and the use of a fluorescent reporter probe. The latter two can be analyzed in real-time.

With agarose gel electrophoresis, the unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown.

The use of SYBR Green dye is more accurate than the agarose gel method, and can give results in real time. A DNA binding dye binds all newly synthesized double stranded DNA and an increase in fluorescence intensity is measured, thus allowing initial concentrations to be determined. However, SYBR Green will label all double-stranded DNA, including any unexpected PCR products as well as primer dimers, leading to potential complications and artifacts. The reaction is prepared as usual, with the addition of fluorescent double-stranded DNA dye. The reaction is run, and the levels of fluorescence are monitored (the dye only fluoresces when bound to the double-stranded DNA). With reference to a standard sample or a standard curve, the double-stranded DNA concentration in the PCR can be determined.

The fluorescent reporter probe method uses a sequence-specific nucleic acid based probe so as to only quantify the probe sequence and not all double stranded DNA. It is commonly carried out with DNA based probes with a fluorescent reporter and a quencher held in adjacent positions (so-called dual-labeled probes). The close proximity of the reporter to the quencher prevents its fluorescence; it is only on the breakdown of the probe that the fluorescence is detected. This process depends on the 5′ to 3′ exonuclease activity of the polymerase involved.

The real-time quantitative PCR reaction is prepared with the addition of the dual-labeled probe. On denaturation of the double-stranded DNA template, the probe is able to bind to its complementary sequence in the region of interest of the template DNA. When the PCR reaction mixture is heated to activate the polymerase, the polymerase starts synthesizing the complementary strand to the primed single stranded template DNA. As the polymerization continues, it reaches the probe bound to its complementary sequence, which is then hydrolyzed due to the 5′-3′ exonuclease activity of the polymerase, thereby separating the fluorescent reporter and the quencher molecules. This results in an increase in fluorescence, which is detected. During thermal cycling of the real-time PCR reaction, the increase in fluorescence, as released from the hydrolyzed dual-labeled probe in each PCR cycle is monitored, which allows accurate determination of the final, and so initial, quantities of DNA.

In some embodiments, use of in situ hybridization is desirable. In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of miRs.

Sample cells or tissues are treated to increase their permeability to allow a probe, such as a miR-specific probe, to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay.

In some embodiments, use of in situ PCR is desirable. In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Use of Differentially-Expressed miRs and miRs as Predictive Markers of Prognosis and for Identification of Therapeutic Agents for Treatment of Lung Cancer

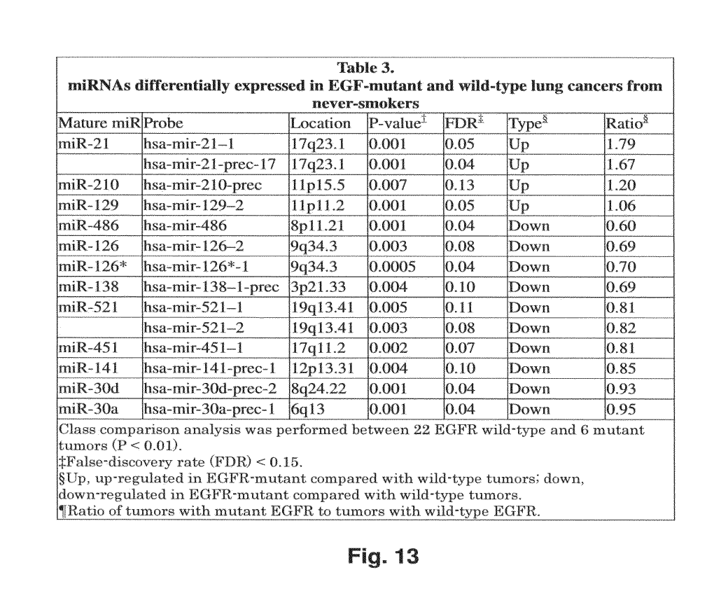

It is disclosed herein that certain expression patterns of miR, along with EGFR mutant status indicators are predictors of survival prognosis in EGFR-mutant patients. EGFR mutant cancer cells samples (for example, tissue biopsy samples) with differentially-expressed miRs (examples of which are shown in FIG. 13—Table 6) when compared to wild type EGFR tumor tissue, predicts a decrease in survival. Thus, the differentially-expressed miR status in tumors can be used as a clinical tool in lung cancer patients' prognosis and treatments. As used herein, “poor prognosis” generally refers to a decrease in survival, or in other words, an increase in risk of death or a decrease in the time until death. Poor prognosis can also refer to an increase in severity of the disease, such as an increase in spread (metastasis) of the cancer to other organs. In one embodiment, the respective markers show at least a 1.5-fold increase or decrease in expression relative to the control. In other embodiments, poor prognosis is indicated by at least a 2-fold, at least a 2.5-fold, at least a 3-fold, at least a 3.5-fold, or at least a 4-fold increase or decrease in the markers relative to the wild-type tumor control figures.

Methods of screening candidate agents to identify therapeutic agents for the treatment of disease are well known in the art. Methods of detecting expression levels of RNA and proteins are known in the art and are described herein, such as, but not limited to, microarray analysis, RT-PCR (including qRT-PCR), in situ hybridization, in situ PCR, and Northern blot analysis. In one embodiment, screening comprises a high-throughput screen. In another embodiment, candidate agents are screened individually.

The candidate agents can be any type of molecule, such as, but not limited to nucleic acid molecules, proteins, peptides, antibodies, lipids, small molecules, chemicals, cytokines, chemokines, hormones, or any other type of molecule that may alter cancer disease state(s) either directly or indirectly. In some embodiments, the candidate agents are molecules that play a role in the NFκB/IL-6 signaling pathway. In other embodiments, the candidate agents are molecules that play a role in the IL-10, STAT3 or interferon-inducible factor signaling networks. In one embodiment, the candidate agents are cytokines. In another embodiment, the candidate agents are small molecules.

Also described herein is a method for the characterization of EGFR mutant never-smoker cancer, wherein at least one feature of EGFR mutant never smoker cancer is selected from one or more of the group comprising: presence or absence of EGFR mutant cancer; diagnosis of EGFR mutant cancer; prognosis of EGFR mutant cancer; therapy outcome prediction; therapy outcome monitoring; suitability of EGFR mutant cancer to treatment, such as suitability of EGFR mutant cancer to chemotherapy treatment and/or radiotherapy treatment; suitability of EGFR mutant cancer to hormone treatment; suitability of EGFR mutant cancer for removal by invasive surgery; suitability of EGFR mutant cancer to combined adjuvant therapy.

Also described herein are kits for the detection of EGFR mutant cancer, the kits can include at least one detection probe comprising a miR or miR herein disclosed as differentially expressed in EGFR mutant cancer. The kit can be in the form or comprises an oligonucleotide array.

Also described herein is a method for the determination of suitability of a EGFR mutant cancer patient for treatment comprising: i) isolating at least one tissue sample from a patient suffering from EGFR mutant cancer; ii) performing the characterization of at least one tissue sample and/or utilizing a detection probe, to identify the EGFR mutant differential miR expression pattern; iii) based on the at least one feature identified in step ii), diagnosing the physiological status of the patient; iv) based on the diagnosis obtained in step iii), determining whether the patient would benefit from treatment of the EGFR mutant cancer. In certain embodiments, the at least one feature of the cancer is selected from one or more of the group comprising: presence or absence of the cancer; type of the cancer; origin of the cancer; diagnosis of cancer; prognosis of the cancer; therapy outcome prediction; therapy outcome monitoring; suitability of the cancer to treatment, such as suitability of the cancer to chemotherapy treatment and/or radiotherapy treatment; suitability of the cancer to hormone treatment; suitability of the cancer for removal by invasive surgery; suitability of the cancer to combined adjuvant therapy.

Also described herein is a method of for the determination of suitability of a cancer for treatment, wherein the at least one feature of the cancer is suitability of the cancer to treatment, such as suitability of the cancer to chemotherapy treatment and/or radiotherapy treatment; suitability of the cancer to hormone treatment; suitability of the cancer for removal by invasive surgery; suitability of the cancer to combined adjuvant therapy.

Also described herein is a method for the determination of the likely prognosis of a cancer patient comprising: i) isolating at least one tissue sample from a patient suffering from cancer; and, ii) characterizing at least one tissue sample to identify the EGFR mutant miR differential expression pattern; wherein the feature allows for the determination of the likely prognosis of the cancer patient.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

### EXAMPLE I

MicroRNA Expression Profiles in Lung Cancers from Never-Smokers

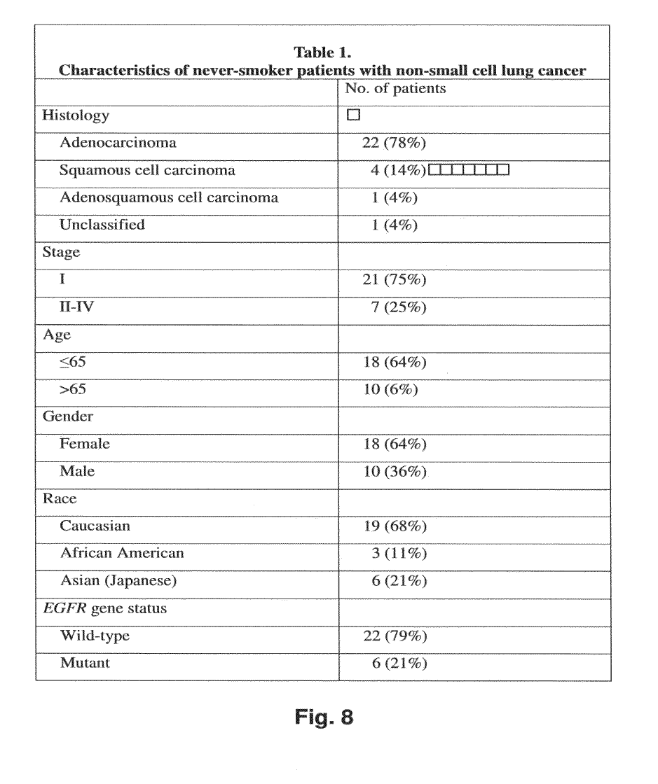

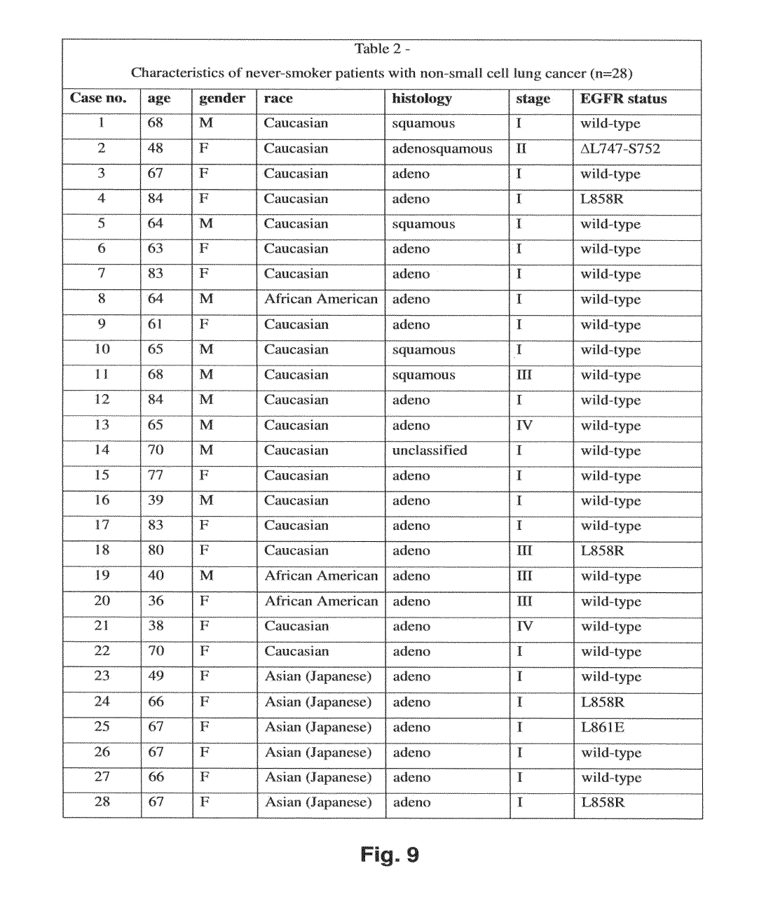

Examined were miR expression profiles in 28 matched pairs of lung cancer and noncancerous lung tissues from never-smokers (FIG. 8—Table 1 and FIG. 9—Table 2) by using the Ohio State miR microarray version 3.0 (21).

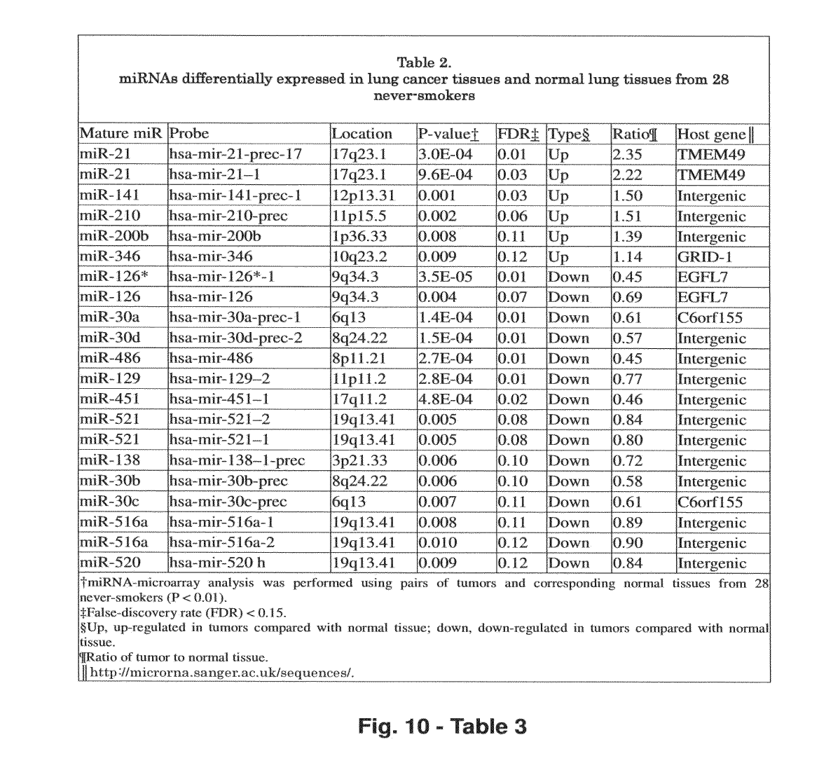

In class comparison analysis using Biometric Research Branch (BRB) array tools, eighteen miRs were found to be differentially expressed in cancers compared to noncancerous tissues (p<0.01 with false discovery rate (FDR)<0.15) (FIG. 10—Table 3).

The expression profiles of these 18 miRs distinguished between cancer and paired noncancerous tissues with the prediction accuracy of 84% using the 3-nearest neighbor algorithm and 82% using the support vector machine algorithm within BRB array tools (10-fold cross validation repeated 100 times).

Five miRs were expressed at higher levels in cancer tissues, with miR-21 enriched the most at 2.35-fold. Expression levels of 13 miRs were lower in cancers, with miR-486 and miR-126* repressed the most at 0.45-fold.

The identification of a single miR by two different probes (miR-21, miR-521 and miR-516a), two mature miRs generated from a single stem-loop pre-miR (miR-126 and miR-126*), and more than one miRs chromosomally clustered and possibly co-regulated (miR-30a and miR-30c on 6q13; miR-30b and miR-30d on 8q24.22; and miR-516a, miR-520 and miR-521 on 19q13.41) all supported the validity of the analysis.

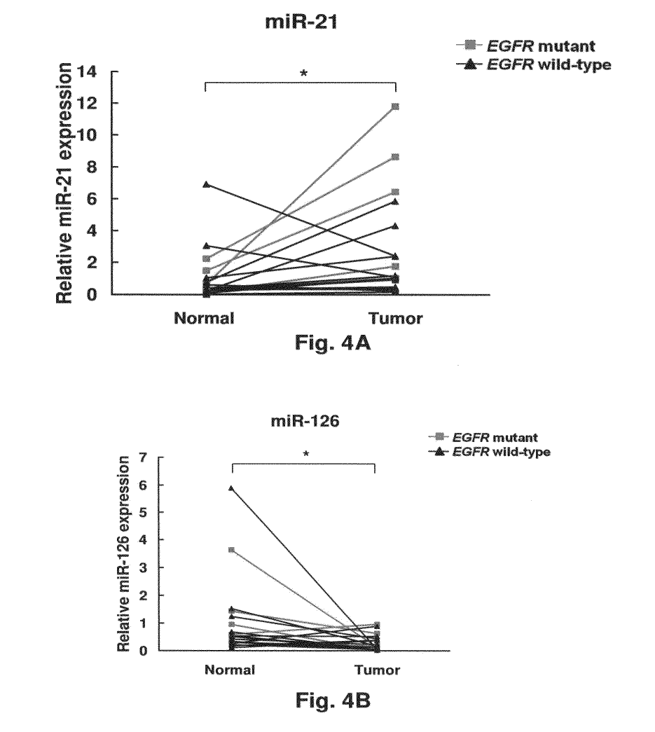

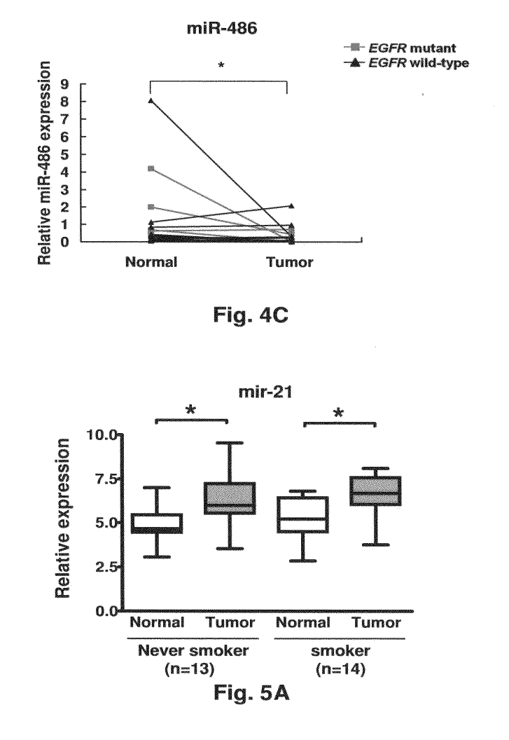

The mRNA microarray data of never-smoker lung adenocarcinoma cases (ncbi.nlm.nih.gov/geo/, accession number=GSE10072) also showed that two host genes, TMEM49 and EGFL7 (FIG. 10—Table 3), were differentially expressed between cancer and noncancerous tissues in the same directions as their resident miRs (miR-21 and miR-126/126*, respectively). The expression levels of three miRs (miR-21, miR-126 and miR-486) were examined by real-time quantitative RT-PCR (qRT-PCR) (FIGS. 4A-4C). MiR-21 expression was significantly higher in cancer tissues than in noncancerous tissues (p<0.05, paired t-test) (FIG. 4A), and miR-126 and miR-486 were expressed at significantly lower levels in cancers (p<0.05, paired t-test, respectively) (FIG. 4B and FIG. 4C), further validating the results of the microarray analysis.

Differential miR Profiles in Lung Cancers from Never-Smokers Versus Smokers

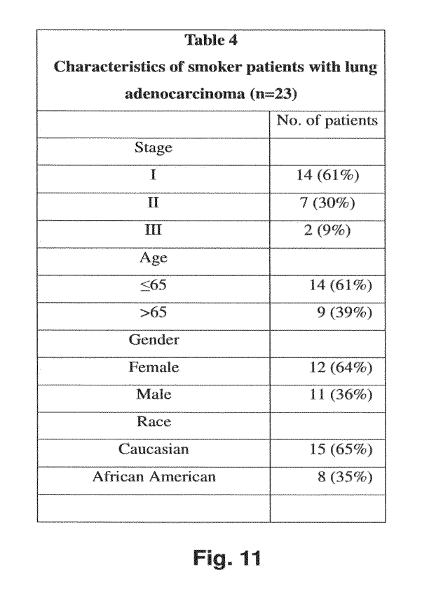

To identify cancer-associated changes in miR expression that are related to smoking status, the inventors compared the miR expression profiles of the present never-smoker cases with those of 58 smoker lung adenocarcinoma cases in the inventors; previous study (Yanaihara N, et al. (2006) Unique microRNA molecular profiles in lung cancer diagnosis and prognosis. Cancer Cell 9:189-198), and additional 23 smoker lung adenocarcinoma cases (FIG. 11—Table 4).

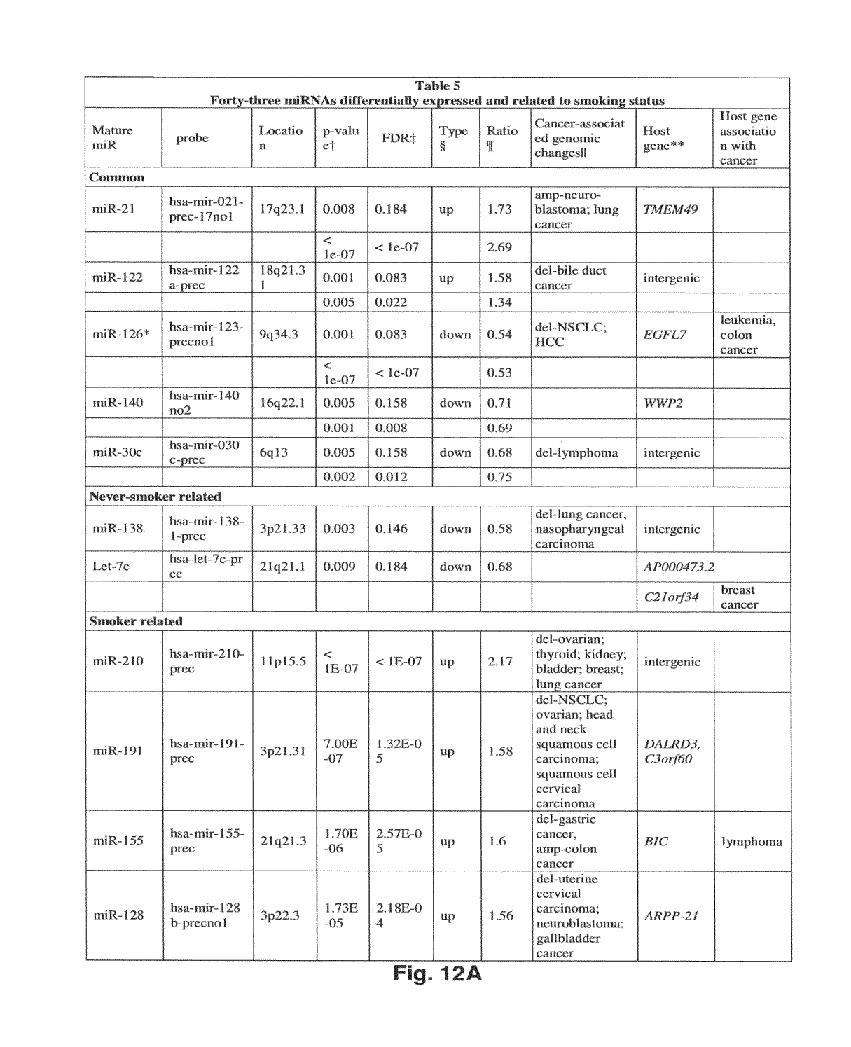

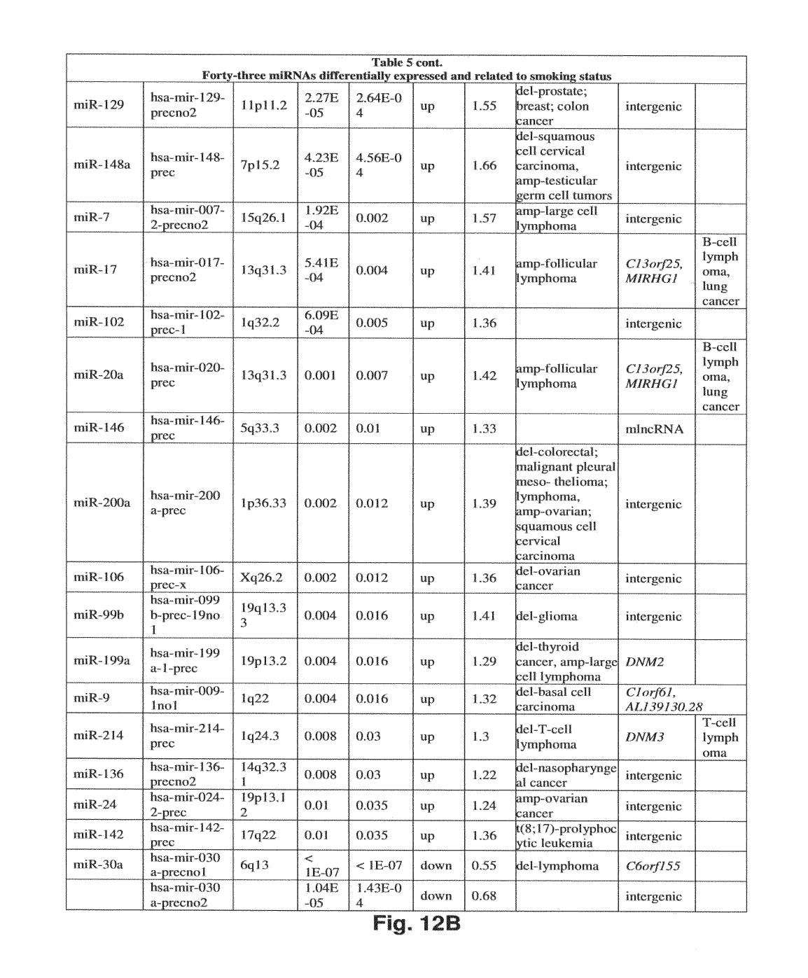

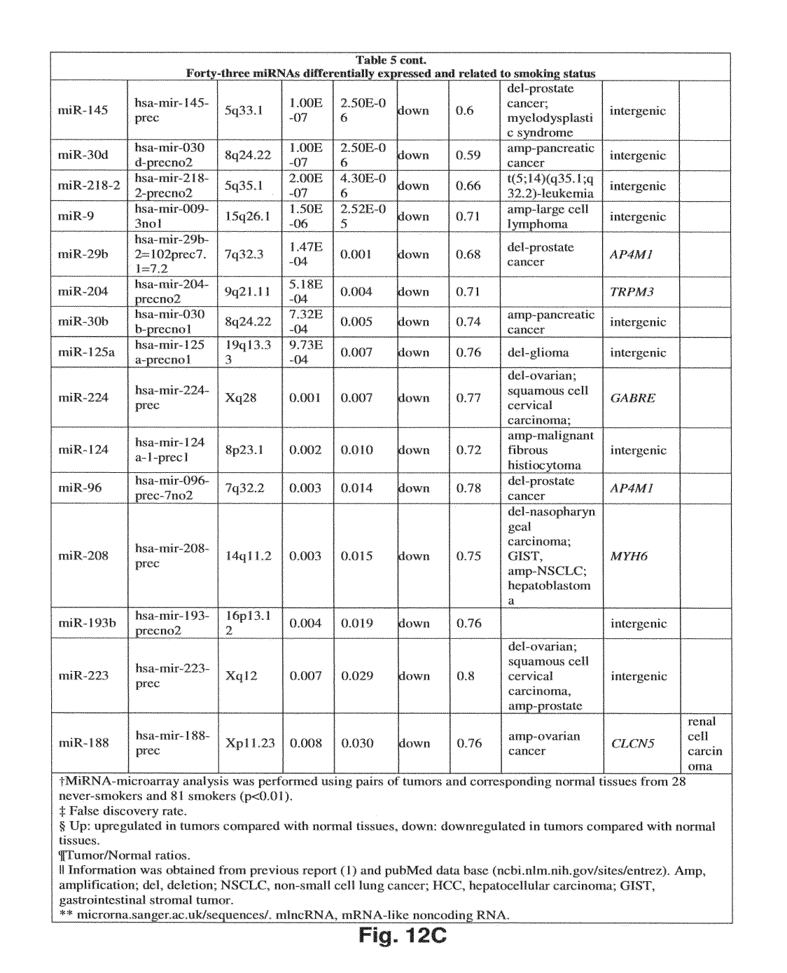

Five miRs were identified to be changed in expression commonly in never-smoker and smoker cases, among which was the increased miR-21 (FIG. 12—Table 5). While only two miRs, miR-138 and let-7c, were significantly changed (both downregulated) in never-smoker cases, altered expression of 36 miRs were preferentially associated with smoker cases (FIG. 12—Table 5), likely reflecting the more extensive genetic and epigenetic changes in smoker-derived lung cancers.

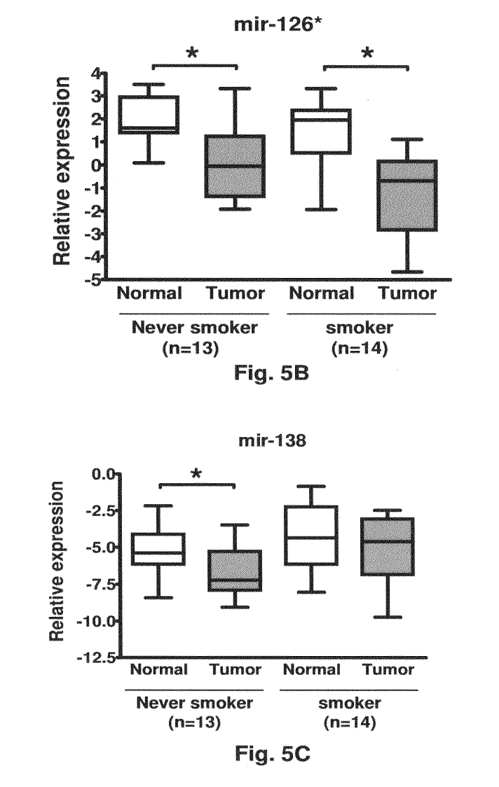

The inventors validated by qRT-PCR the specific downregulation of miR-138 in never-smoker adenocarcinomas, as well as the upregulation of miR-21 and the downregulation of miR-126* irrespective of smoking status (FIG. 5). Interestingly, miR-138 is located at chromosome 3p21.33, a candidate locus that carries a lung cancer suppressor gene, and was reported to target the human telomerase reverse transcriptase (hTERT) gene, on which a variety of cellular and viral oncogenic mechanisms act. A role of this miR in etiology of lung cancers from never-smokers deserves further investigation.

MiR Expression Profiles Associated with EGFR Gene Mutations

The status of EGFR gene was determined by DNA sequencing in the 28 lung cancer tissues from never-smokers (FIG. 9—Table 2). Six cases were found to have the activating mutations of EGFR in the tyrosine kinase domain: four with an amino acid substitution from leucine to arginine at codon 858 (L858R); one with an amino acid substitution from leucine to glutamate at codon 861 (L861E); and one with an in-frame deletion of codons 747 to 752 (ΔL747-S752). The class comparison analysis of miR expression between 22 EGFR wild-type and 6 EGFR mutant cases found twelve miRs that were significantly more or less abundant in EGFR mutant cases (p<0.01 with FDR<0.15) (FIG. 13—Table 6).

Ten out of the 12 miRs (miR-21, miR-210, miR-486, miR-126, miR-126*, miR-138, miR-521, miR-451, miR-30d and miR-30a) were above identified in the comparison of cancer versus noncancerous tissues to be changed in the same directions (FIG. 10—Table 3), showing that EGFR mutations may reinforce the aberrant regulation of some miRs associated with lung carcinogenesis in never-smokers. MiR-21 and miR-486, which were most upregulated and downregulated in cancer versus noncancerous tissues, respectively, again showed the most difference between EGFR mutant and wild-type cancers (˜1.7-fold and 0.60-fold, respectively). Although the qRT-PCR data shown in FIG. 4 were performed on a limited number of cases, thereby limiting our ability to show a statistically significant difference between EGFR mutant and wild-type cases in any of miR-21, miR-126 or miR-486 expression, it should be noted that three cases expressing the highest levels of miR-21 in cancer (cases 24, 25 and 28) all had the activating mutation of EGFR (FIG. 4 and FIG. 9—Table 2).

Expression of miR-21 and the Status of EGFR Signaling in Lung Cancer Cell Lines

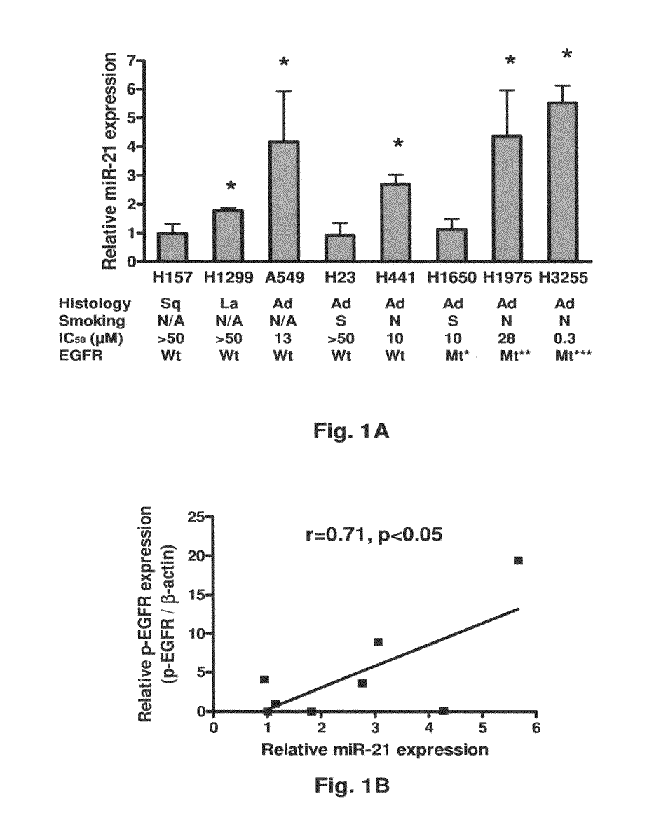

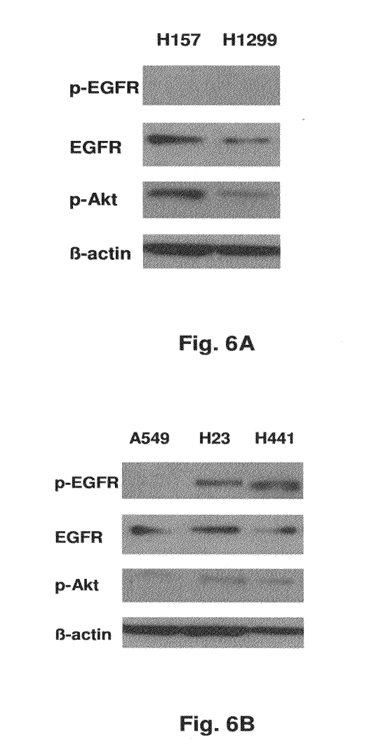

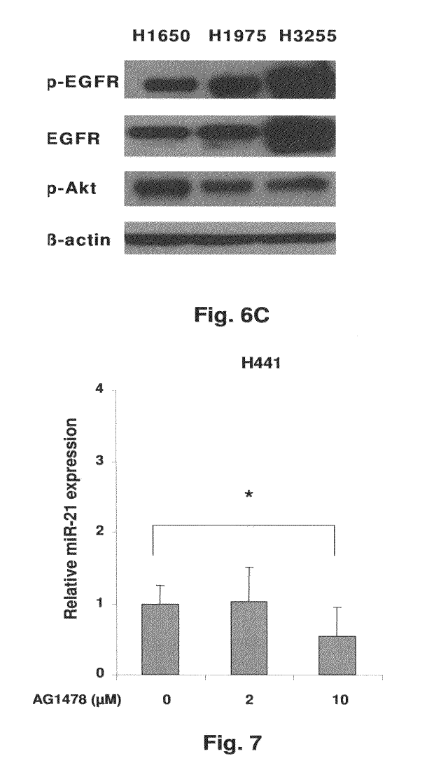

Because of its most remarkable increase in cancer versus noncancerous tissues and its association with EGFR mutations, an indicator of sensitivity to EGFR-TKIs, miR-21 was chosen for further analyses. To investigate a correlation between miR-21 expression levels and the status of EGFR signaling pathway, eight non-small cell lung cancer (NSCLC) cell lines were examined in Western blot (FIGS. 6A, 6B and 6C) and qRT-PCR analyses (FIG. 1A).

Among them, three adenocarcinoma cell lines were mutant for EGFR: H3255 with L858R; H1975 with both L858R and a substitution from threonine to methionine at codon 790 (1790M); and H1650 with an in-frame deletion of codons 746 to 750 (ΔE746-A750). These three EGFR mutant cell lines had high levels of phosphorylated EGFR (p-EGFR), as well as increased amounts of total EGFR protein and induction of phosphorylated Akt (p-Akt) (FIG. 6C), consistent with the constitutive activation of EGFR signaling pathway in these cells. Two of the three (H3255 and H1975), but not the third cell line (H1650), expressed elevated levels of miR-21 (FIG. 1A).

Three out of five EGFR wild-type cell lines, either with (H441) or without (A549 and H1299) detectable levels of p-EGFR (FIGS. 6A and 6B), also expressed significantly higher levels of miR-21 than control untransformed cells (FIG. 1A). The quantitative comparison of miR-21 and pEGFR levels showed a significant positive correlation between these two (Pearson's correlation, r=0.71, p<0.05) (FIG. 1B). These results suggest that the activated EGFR signaling pathway is a major, but not sole, mechanism of miR-21 regulation. It was also noteworthy to find that miR-21 expression and/or EGFR status correlated with sensitivity to an EGFR-TKI, AG1478, which was indicated as half maximal inhibitory concentration (IC50) (FIG. 1A): the five cell lines showing AG1478-inhibited cell proliferation had either mutant EGFR (H1650) or expressed >2-fold increased levels of miR-21 (H441 and A549), or both (H3255 and H1975). Two lung adenocarcinoma cell lines derived from never-smoker cancers were selected for the functional assays of miR-21 (see below): H3255 with high sensitivity to AG1478 (IC50, 0.3 μM), mimicking never-smoker lung cancer cases with mutant EGFR and highest levels of miR-21 (e.g., case numbers 24, 25 and 28 in FIG. 4A and FIG. 9—Table 2); and H441 with intermediate sensitivity to AG1478 (IC50, 10 μM), mimicking ones with wild-type EGFR but still with significantly increased levels of miR-21 (e.g., case numbers 5 and 23 in FIG. 4A and FIG. 9—Table 2).

Activated EGFR Signaling Enhances miR-21 Expression

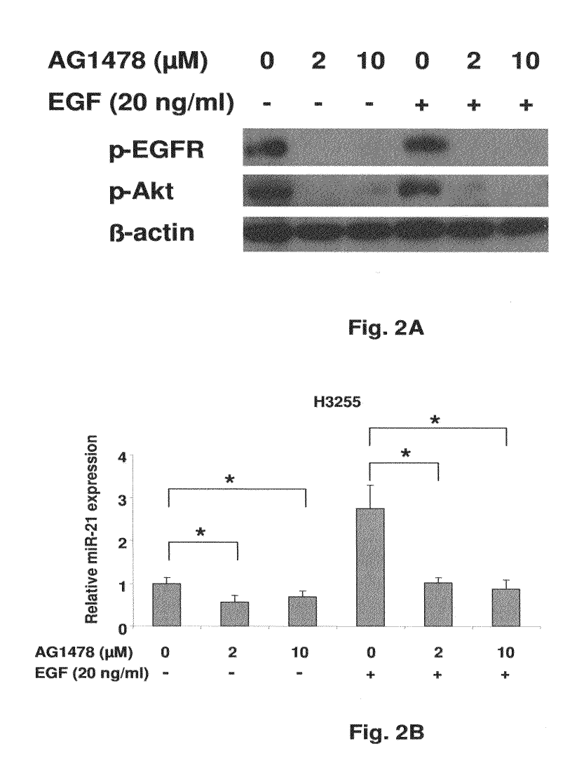

To experimentally verify whether the activated EGFR signaling is responsible for elevated levels of miR-21 expression, EGFR mutant H3255 cells were treated with AG1478 in the presence or absence of EGF (FIG. 2).

AG1478 at either 2 μM or 10 μM effectively inhibited the EGFR signaling under conditions with or without EGF ligand stimulation, as shown by diminished p-EGFR and p-Akt (FIG. 2A), consistent with the IC50 value of 0.3 μM in this cell line. The levels of miR-21 expression in the absence of EGF were significantly repressed by treatment with either concentration of AG1478 (p<0.01, paired t-test) (FIG. 2B, left). The addition of EGF resulted in ˜2.5-fold upregulation of miR-21 expression, which was still inhibited back to the basal levels by AG1478 treatment with either concentration (p<0.05, paired t-test) (FIG. 2B, right).

These results indicate that miR-21 expression is positively regulated by the activated EGFR signaling in cancer cells with an activating EGFR mutation, and that EGFR-TKIs can effectively repress the aberrantly increased miR-21. In H441 cells with wild-type EGFR, AG1478 at 10 μM (equivalent to the IC50 value in this cell line), but not at 2 μM, significantly repressed miR-21 expression (p<0.05, paired t-test) (FIG. 7).

Thus, the activated signaling from wild-type EGFR in H441 cells (FIG. 6B), likely through a self-produced transforming growth factor (TGF)-alpha stimulation, can also be inhibited by AG1478, resulting in the repression of miR-21.

Antisense Inhibition of miR-21 Induces Apoptosis in Cooperation with EGFR-TKI

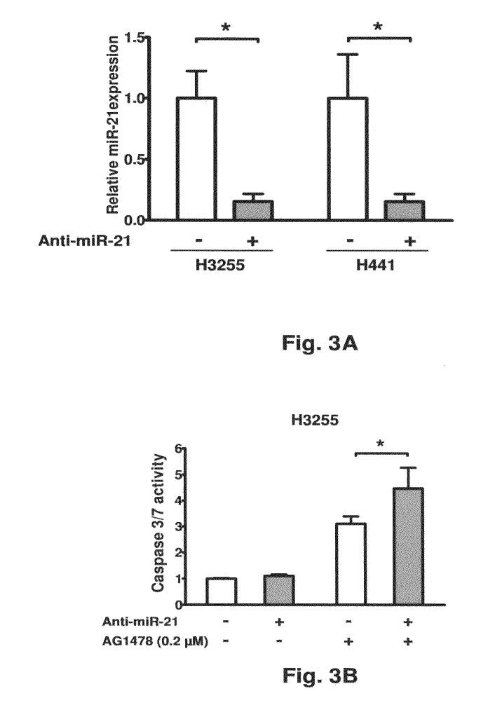

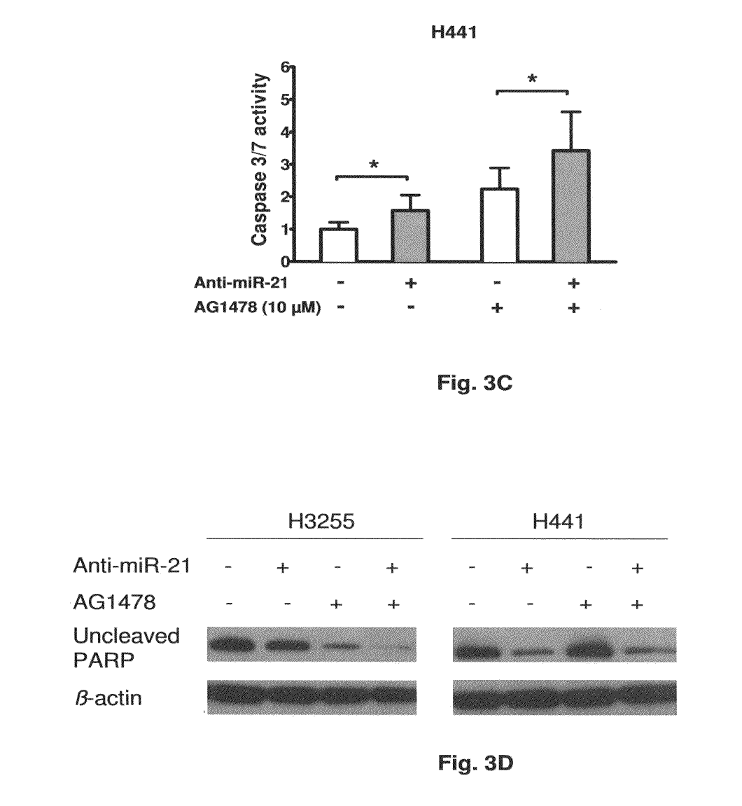

To examine the biological activity of elevated miR-21 expression in never-smoker-derived lung cancer, H3255 and H441 cells were transfected with an antisense oligonucleotide targeting miR-21 (anti-miR-21). The antisense-mediated repression of miR-21 in these cells was confirmed by qRT-PCR (FIG. 3A). As miR-21 reportedly has an anti-apoptotic activity, the inventors herein determined whether inhibition of miR-21 induces apoptosis in these cells by an assay measuring caspase-3 and caspase-7 enzymatic activities (FIGS. 3B and 3C). In H3255 cells, anti-miR-21 alone did not induce apoptosis (FIG. 3B, left). Notably, however, when used in combination with AG1478 at 0.2 μM (a concentration slightly lower than the IC50 value), anti-miR-21 significantly enhanced AG1478-induced apoptotic response (FIG. 3B, right). In H441 cells, anti-miR-21 by itself resulted in a significant increase in apoptotic response (FIG. 3C, left), although it was less effective than AG1478 treatment at 10 μM (a concentration equivalent to the IC50 value). Similar to the combinational effect observed in H3255 cells, anti-miR-21 further enhanced apoptotic response induced by 10 μM of AG1478 in H441 cells (FIG. 3C, right).

The effect of anti-miR-21 on apoptosis was further substantiated by Western blot analysis of poly(ADP-ribose) polymerase (PARP), a main cleavage target of caspase-3 in the apoptotic response (FIG. 3D). The amounts of uncleaved PARP were markedly decreased in H3255 cells treated with both anti-miR-21 and AG1478 and in H441 cells treated with anti-miR-21 in the presence or absence of AG1478, where anti-miR-21 caused significant increases in caspase 3/7 activities.

Discussion of Example I

Example I now, for the first time, clarifies miR expression profiles in lung cancer in never-smokers. By comparing the profiles with those of smoker cases and analyzing the data according to the status of the EGFR gene, the Example I shows novel molecular signatures of lung cancers in never-smokers:

1) changes in expression of a relatively small number of miRs are involved in lung carcinogenesis in never-smokers;

2) EGFR mutations may reinforce some of these changes in miR expression, e.g., an increase in miR-21;

3) miR-138 on 3p21.33, a chromosomal region carrying a long-sought lung cancer suppressor gene, is downregulated preferentially in never-smoker cases; and

4) miR-21 is one of the most aberrantly increased miRs in both never-smoker and smoker cases.

These findings identified miR-21 as playing an oncogenic role in lung carcinogenesis. Therefore, the inventors chose it as a candidate for novel molecular targets in treatment of lung cancers in never-smokers, as well as those in smokers. While not wishing to be bound by theory, given the relationship between EGFR mutations and miR-21 upregulation, the inventors herein now believe that this miR has implications in improving EGFR-TKI therapy, whose effectiveness is correlated with EGFR gene status and smoking history of the patients.

Although high levels of miR-21 expression are found in various types of human tumors, including lung cancer from both smokers and never-smokers (present invention), it is not well understood what mechanism upregulates miR-21 during carcinogenesis.

In addition to the miR microarray data showing higher levels of miR-21 in EGFR mutant cases (FIG. 13—Table 6), the in vitro analyses using NSCLC cell lines showed that the activated EGFR signaling upregulates miR-21 expression. A statistically significant positive correlation was observed between miR-21 expression levels and p-EGFR levels in NSCLC cell lines (FIG. 1B). Furthermore, the treatment with the EGFR-TKI (AG1478) inhibited miR-21 expression in two NSCLC cell lines with elevated p-EGFR, EGFR mutant H3255 (FIG. 2) and EGFR wild-type H441 (FIG. 7), providing a mechanistic link between the activated EGFR signaling pathway and the aberrant upregulation of miR-21 and a therapeutic basis for inhibition of miR-21 in lung cancers with EGFR activation. STAT3, which reportedly signals IL6-induced upregulation of miR-21 in multiple myeloma cells, or increased p-Akt (FIG. 2A and FIG. 6) can mediate the EGFR signaling-induced upregulation of miR-21. Nevertheless, high levels of miR-21 in A549 cells without EGFR mutation or p-EGFR (FIG. 1A and FIG. 6B) and no increased miR-21 expression in H1650 cells with EGFR mutation and increased p-EGFR (FIG. 1A and FIG. 6C) suggest that there should also be EGFR-independent mechanisms to control miR-21 expression.

Antisense oligonucleotide-mediated knockdown was successfully performed to inhibit miR-21 expression in H3255 and H441 (FIG. 3A), two NSCLC cell lines likely recapitulating some lung cancer cases from never-smokers, which expressed elevated levels of miR-21 in the presence or absence of EGFR mutation (FIG. 4A). The antisense-inhibition of miR-21 by itself led to increased apoptotic response in H441 cells (FIG. 3C and FIG. 3D), suggesting that miR-21 can be a therapeutic target in lung cancers as well. Importantly, in both cell lines, anti-miR-21 significantly enhanced the apoptotic response induced by AG1478 (FIG. 3B and FIG. 3C). No effect of anti-miR-21 alone in H3255 cells (FIG. 3B) may suggest that the combinational use of anti-miR-21 and EGFR-TKI is required to effectively attenuate the constitutively activated EGFR signaling pathway to cell survival, which is evidenced by the highest levels of p-EGFR (FIG. 3C) and miR-21 (FIG. 1A). While EGFR-TKIs are widely in clinical use for lung cancer and inhibition of oncogenic miRs is a new promising approach in cancer therapy, the Example I for the first time reveals that the combination of these two therapeutic strategies can be significantly more effective than either alone. The finding is of particular importance in developing better treatment for lung cancer patients of non-Asian ethnicity, who tend to be less responsive to the current EGFR-TKI therapy. The Example I also illustrates the usefulness in preventing and rescuing acquired EGFR-TKI resistance in NSCLC, an important issue of clinical relevance. Besides a secondary T790M mutation and acquired MET amplification, selection of an EGFR wild-type subpopulation on a background of wild-type/mutant mixture leads to acquired EGFR-TKI resistance in NSCLC. The combinatorial use of EGFR-TKI and anti-miR-21 can be used to prevent and rescue such acquired resistance due to selection for wild-type EGFR, as anti-miR-21 is effective on both EGFR wild-type and mutant tumor cells. Recently, intravenous administration of locked nucleic acid-modified oligonucleotides (LNA-anti-miR) antagonized the liver-expressed miR-122 in primates, supporting the feasibility of in vivo targeting miRs in therapy of human diseases.

Thus, Example I shows that lung cancers in never-smokers have unique miR expression profiles as a novel molecular characteristic distinct from lung cancers in smokers. MiR-21 is a downstream effector of the activated EGFR signaling pathway and can be a therapeutic target in lung cancers with and without EGFR mutations. Antisense inhibition of miR-21 can be useful to improve clinical response to EGFR-TKI therapy.

Materials and Methods for Example I

Clinical Samples

Twenty-eight pairs of lung cancer tissues and corresponding noncancerous lung tissues were obtained from never-smokers who had undergone surgical resection from 2000 to 2004 at the University of Maryland Medical Center (n=15), Mayo Clinic (n=7) in the United States and Hamamatsu University School of Medicine (n=6) in Japan (Table 1 and S1). All tissues were freshly collected during surgery, snap-frozen, and stored at −80° C. Twenty-one patients had stage I disease, one had stage II disease, four had stage III disease, and two had stage IV disease according to World Health Organization TNM (tumor-node-metastasis) staging. Twenty-two cases were EGFR wild-type and six cases were EGFR mutant (FIG. 8—Table 1 and FIG. 9—Table 2). Institutional review board approval and written informed consent from all patients were obtained at each collection site.

Cell Culture

Six lung adenocarcinoma cell lines (A549, H23, H441, H1650, H1975 and H3255), one squamous cell line (H157) and one large cell carcinoma cell line (H1299) were used in this study. H3255 was provided by National Cancer Institute and maintained in ACL-4 medium (GIBCO) with 5% fetal bovine serum (GIBCO). A549, H23, H441, H1650, H1975, H157 and H1299 were purchased from American Type Culture Collection (ATCC) and maintained in RPMI 1640 (GIBCO) with 10% fetal bovine serum. hTERT-immortalized normal human bronchial epithelial cells (HBET2) were established.

Microarray Analysis

Total RNA was isolated by TRIzol reagent (Invitrogen, Carlsbad, Calif.), according to the manufacturer's instructions. Microarray analysis was performed as previously described. Briefly, 5 μg of total RNA was used for hybridization on miR microarray chips containing 389 probes in triplicate (Ohio State microRNA microarray version 3.0, Columbus, Ohio). Processed slides were scanned using a PerkinElmer ScanArray XL5K Scanner. Using R, only spot values that were not flagged by the image quantification software GenePix Pro 6.0.1.00 and whose foreground intensities were larger than background intensities were used. The remaining spots were then LOESS (LOcally wEighted Scatterplot Smoothing) normalized and duplicate spots were averaged. The preprocessed and normalized data was then imported into BRB-ArrayTools version 3.5.0 (linus.nci.nih.gov/BRB-ArrayTools.html). Finally, 291 miRs with non-missing log values present in more than 75% of the samples were selected.

Real-Time RT-PCR Analysis

Expression of mature miRs was examined using qRT-PCR analysis using a TaqMan Human MicroRNA Assay kit (Applied Biosystems, Foster City, Calif.). RNU6B was used as an endogenous control (#4373381, Applied Biosystems). Reactions were performed using a PRISM 7700 Sequence Detector System (Applied Biosystems). Gene expression was quantified and values were reported as 2-ΔΔCT. Data were presented as mean±SD from triplicate.

Cell Treatment and Growth Inhibition Assay

AG1478 was purchased from Calbiochem (San Diego, Calif.). Epidermal growth factor (EGF) was purchased from Promega (Madison, Wis.). To evaluate the effect of AG1478 on the EGFR signaling pathway and miR-21 expression levels, lung cancer cell lines were serum-starved for 24 h, incubated in the presence or absence of AG1478 (2 μM or 10 μM) for 2 h, and then for an additional 15 min in the presence or absence of EGF (20 ng/ml).

Growth inhibition was assessed by MTS assay (Dojindo, Japan) to examine the effect of AG1478 on lung cancer cell lines. Cell suspensions (5,000 cells/well) were seeded into 96-well plates and increasing concentrations of AG1478 (0, 0.4, 2.0, 10 and 50 μM) were added. After incubation for 72 h at 37° C., MTS [3-(4,5-dimethylthiazol-2-yl)-5-(3-carboxymethoxyphenyl)-2-(4-sulfophenyl) 2H-tetrazolium, inner salt] was added to each well and incubated for 2 h at 37° C., and then absorbance was measured using a microplate reader with a test wavelength of 450 nm. The IC50 value was defined as the concentration needed for 50% reduction of the growth by treatment with AG1478. Each experiment was done at least in triplicate, and four times independently. The data were shown as mean±SD.

Antibodies and Western Blot Analysis

Cells were lysed in buffer containing 50 mM Tris-HCl, pH 7.6, 150 mM NaCl, 0.1% sodium dodecyl sulfate, 1% Nonidet P-40, and 0.5% sodium-deoxycholate. The lysates were kept on ice for 30 min, and then centrifuged at 13000 g for 30 min. The supernatant was collected and then 10 μg of protein were separated by gel electrophoresis on 10% gels, transferred to nitrocellulose membranes and detected by immunoblotting using a chemiluminescence system (GE Healthcare Bio-Sciences Corp, Piscataway, N.J.). The images were quantified by measuring signal intensity using NIH ImageJ1.40g (rsb.info.nih.gov/ij/). The antibodies detecting EGFR, phospho-EGFR (Tyr1173), phospho-Akt (Ser473), PARP and β-actin were purchased from Cell Signaling Technology (Beverley, Mass.).

Oligonucleotide Transfection and Apoptosis Assay

2′-O-methyl oligonucleotides were chemically synthesized at Integrated DNA Technologies (Coralville, Iowa). 2′-O-methyl oligonucleotides had the following sequences: 2′OMe-enhanced green fluorescent protein (EGFP) (anti-EGFP) 5′-AAG GCA AGC UGA CCC UGA AGU-3′ [SEQ ID NO:1] and 2′OMe-miR-21 (anti-miR-21) 5′-UCA ACA UCA GUC UGA UAA GCUA-3′ [SEQ ID NO:2]. H441 and H3255 cells were plated in triplicate in 96-well plates. Cells were transfected using LipofectAMINE 2000 reagent (Invitrogen) 24 h after plating. Transfection complexes were prepared according to the manufacturer's instructions and added directly to the cells to a final oligonucleotide concentration of 40 nM. Transfection medium was replaced 8 h post-transfection. After 72 h, the cells were incubated in the presence or absence of 0.2 μM of AG1478 for 24 h (H3255) or 10 μM for 72 h (H441). Activities of caspase-3 and caspase-7 were analyzed using ApoONE Homogeneous Caspase 3/7 Assay (Promega) according to the manufacturer's instructions. Samples were measured after 6 h of incubation with the caspase substrate on a fluorescent plate reader using wavelengths of 485 and 535 nm for excitation and emission, respectively. Each experiment was done in triplicate, and at least four times independently. The data were shown as mean±SD.

Statistical Analysis:

Paired t-test identified differentially expressed miRs between lung cancer tissues and normal lung tissues (p<0.01, FDR<0.15). We also identified miRs that were differently expressed between EGFR mutant and wild-type lung cancers using F-test (p<0.01, FDR<0.15). Paired t-test was used to analyze differences in miR expression (miR-21, miR-126 and miR-486) between tumors and corresponding normal tissues for qRT-PCR data. Graphpad Prism v5.0 (Graphpad Softoware Inc, La Jolla, Calif.) analysis was used for the Pearson's correlation. All statistical tests were two-sided, and statistical significance was defined as P<0.05.

### EXAMPLE II

MiR Profiling Comparison of Lung Cancers from Never-Smokers and Smokers

Ohio State miR microarray data of the present 28 never-smoker cases (version 3.0) and those of 58 smoker lung adenocarcinoma cases in our previous study (version 1.0) with additional 23 smoker cases (version 2.0) (FIG. 11—Table 4) were analyzed. Expression data comprising only the probes that were in common among all versions were LOESS normalized within each version group using R. Next, z-scores were calculated within each version and data from all versions was merged. The merged dataset was then imported into BRB-ArrayTools version 3.5.0 to identify differentially expressed miRs (p<0.01, FDR<0.2).

mRNA Expression Data of Host Genes

Messenger RNA microarray data of 20 never-smoker lung adenocarcinoma cases were downloaded from GEO database (ncbi.nlm.nih.gov/geo/, GSE10072) and analyzed by BRB-ArrayTools version 3.5.0.

### EXAMPLE III

Methods of Treating Lung Cancer Patients

This example describes a method of selecting and treating patients that are likely to have a favorable response to treatments with compositions herein.

A patient diagnosed with lung cancer ordinarily first undergoes lung resection with an intent to cure. Lung tumor samples are obtained from the portion of the lung tissue removed from the patient. RNA is then isolated from the tissue samples using any appropriate method for extraction of small RNAs that are well known in the art, such as by using TRIZOL™. Purified RNA is then subjected to RT-PCR using primers specific miR21 or other differentially expressed miRs disclosed in FIG. 13—Table 6, optionally in conjunction with EGFR genetic analysis or EGFR phosphorylization analysis. These assays are run to determine the expression level of the pertinent RNA in the tumor. If differentially expressed miR expression pattern is determined, especially if EGFR mutant status is ascertained, the patient is a candidate for treatment with the compositions herein.

Accordingly, the patient is treated with a therapeutically effective amount of the compositions according to methods known in the art. The dose and dosing regimen of the compositions will vary depending on a variety of factors, such as health status of the patient and the stage of the lung cancer. Typically, treatment is administered in many doses over time.

### EXAMPLE IV

Methods of Diagnosing EGFR Mutant Lung Cancer Patients

In one particular aspect, there is provided herein a method of diagnosing whether a subject has, or is at risk for developing, EGFR mutant lung cancer. The method generally includes measuring the differential miR expression pattern of the miRs in FIG. 13—Table 6, especially miR-21 upregulation compared to control. If a differential miR expression pattern is ascertained, the results are indicative of the subject either having, or being at risk for developing, EGFR mutant lung cancer. In certain embodiments, the level of the at least one gene product is measured using Northern blot analysis. Also, in certain embodiments, the level of the at least one gene product in the test sample is less than the level of the corresponding miR gene product in the control sample, and/or the level of the at least one miR gene product in the test sample is greater than the level of the corresponding miR gene product in the control sample.

### EXAMPLE V

Measuring miR Gene Products

The level of the at least one miR gene product can be measured by reverse transcribing RNA from a test sample obtained from the subject to provide a set of target oligodeoxynucleotides; hybridizing the target oligodeoxynucleotides to a microarray comprising miR-specific probe oligonucleotides to provide a hybridization profile for the test sample; and, comparing the test sample hybridization profile to a hybridization profile generated from a control sample. An alteration in the signal of at least one miR is indicative of the subject either having, or being at risk for developing, lung cancer, particularly EGFR mutant lung cancer.

### EXAMPLE VI

Diagnostic and Therapeutic Applications

In another aspect, there is provided herein are methods of treating an EGFR mutant lung cancer in a subject, where the signal of at least one miR, relative to the signal generated from the control sample, is de-regulated (e.g., down-regulated and/or up-regulated).

Also provided herein are methods of diagnosing whether a subject has, or is at risk for developing, a EGFR mutant lung cancer associated with one or more adverse prognostic markers in a subject, by reverse transcribing RNA from a test sample obtained from the subject to provide a set of target oligodeoxynucleotides; hybridizing the target oligodeoxynucleotides to a microarray comprising miR-specific probe oligonucleotides to provide a hybridization profile for the test sample; and, comparing the test sample hybridization profile to a hybridization profile generated from a control sample. An alteration in the signal is indicative of the subject either having, or being at risk for developing, the cancer.

Also provided herein are methods of treating EGFR mutant lung cancer in a subject who has EGFR mutant lung cancer in which at least two miR gene products of miRs of Table 6 are down-regulated or up-regulated in the cancer cells of the subject relative to control cells. When the at least two gene products are down-regulated in the cancer cells, the method comprises administering to the subject an effective amount of at least two isolated gene products, such that proliferation of cancer cells in the subject is inhibited. When two or more gene products are up-regulated in the cancer cells, the method comprises administering to the subject an effective amount of at least one compound for inhibiting expression of at least one gene product, such that proliferation of cancer cells in the subject is inhibited. Also provided herein are methods of treating EGFR mutant lung cancer in a subject, comprising: determining the amount of at least two miR (indicated in FIG. 13—Table 6) gene products in EGFR mutant lung cancer cells, relative to control cells; and, altering the amount of the gene products expressed in the EGFR mutant lung cancer cells by: administering to the subject an effective amount of at the at least two gene products, if the amount of the gene products expressed in the cancer cells is less than the amount of the gene products expressed in control cells; or administering to the subject an effective amount of at least one compound for inhibiting expression of the at least two gene products, if the amount of the gene product expressed in the cancer cells is greater than the amount of the gene product expressed in control cells, such that proliferation of cancer cells in the subject is inhibited.

### EXAMPLE VII

Compositions

Also provided herein are pharmaceutical compositions for treating EGFR mutant lung cancer, comprising at least two isolated miR (indicated in FIG. 13—Table 6) gene products and a pharmaceutically-acceptable carrier. In a particular embodiment, the pharmaceutical compositions comprise gene products corresponds to gene products that are down-regulated in EGFR mutant lung cancer cells relative to suitable control cells.

In another particular embodiment, the pharmaceutical composition comprises at least one expression regulator (for example, an inhibitor) compound and a pharmaceutically-acceptable carrier.

Also provided herein are pharmaceutical compositions that include at least one expression regulator compound that is specific for a gene product that is up- or down-regulated in EGFR mutant lung cancer cells relative to suitable control cells.

### EXAMPLE VIII

Kits

Any of the compositions described herein may be comprised in a kit. In a non-limiting example, reagents for isolating miR, labeling miR, and/or evaluating a miR population using an array are included in a kit. The kit may further include reagents for creating or synthesizing miR probes. The kits will thus comprise, in suitable container means, an enzyme for labeling the miR by incorporating labeled nucleotide or unlabeled nucleotides that are subsequently labeled. It may also include one or more buffers, such as reaction buffer, labeling buffer, washing buffer, or a hybridization buffer, compounds for preparing the miR probes, and components for isolating miR. Other kits may include components for making a nucleic acid array comprising oligonucleotides complementary to miRs, and thus, may include, for example, a solid support.

For any kit embodiment, including an array, there can be nucleic acid molecules that contain a sequence that is identical or complementary to all or part of any of the sequences herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miR from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled miR. It may also include components that preserve or maintain the miR or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of miR.

It is also contemplated that any embodiment discussed in the context of a miR array may be employed more generally in screening or profiling methods or kits of the invention. In other words, any embodiments describing what may be included in a particular array can be practiced in the context of miR profiling more generally and need not involve an array per se.

It is also contemplated that any kit, array or other detection technique or tool, or any method can involve profiling for any of these miRs. Also, it is contemplated that any embodiment discussed in the context of a miR array can be implemented with or without the array format in methods of the invention; in other words, any miR in a miR array may be screened or evaluated in any method of the invention according to any techniques known to those of skill in the art. The array format is not required for the screening and diagnostic methods to be implemented.

The kits for using miR arrays for therapeutic, prognostic, or diagnostic applications and such uses are contemplated by the inventors herein. The kits can include a miR array, as well as information regarding a standard or normalized miR profile for the miRs on the array. Also, in certain embodiments, control RNA or DNA can be included in the kit. The control RNA can be miR that can be used as a positive control for labeling and/or array analysis.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

### EXAMPLE IX

Array Preparation and Screening

Also provided herein are the preparation and use of miR arrays, which are ordered macroarrays or microarrays of nucleic acid molecules (probes) that are fully or nearly complementary or identical to a plurality of miR molecules or precursor miR molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miR-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect miR; consequently, methods and compositions may be used with a variety of different types of miR arrays.

In view of the many possible embodiments to which the principles of our invention may be applied, it should be recognized that the illustrated embodiments are only preferred examples of the invention and should not be taken as a limitation on the scope of the invention. Rather, the scope of the invention is defined by the following claims. We therefore claim as our invention all that comes within the scope and spirit of these claims.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A composition of matter comprising at least one anti-sense miR and at least one additional composition,
wherein the anti-sense miR is anti-sense to a miR that is differentially expressed in epidermal growth factor receptor (EGRF) never-smoker mutant cancer cells compared to wild-type never-smoker cancer cells, and wherein the at least one additional composition is useful to treat cancer.

2. A composition of claim 1, wherein the at least one additional composition is selected from the group comprising: a chemotherapy drug; AG1478; gefitinib (Iressa®); erlotinib (Tarceva®); cetuximab; panitumab; zalutumamab; nimotuzamab; matuzumab; and lapatinib.

3. A composition of claim 1, wherein the anti-sense miR is selected from a miR that is anti-sense to a miR selected from the group: miR-21; miR-210; miR-129.

4. A composition of claim 1, wherein the at least one anti-sense miR is anti-sense to miR-21.

5. A composition of claim 4, wherein the at last one additional composition useful to treat cancer is an epidermal growth factor receptor tyrosine kinase inhibitor.

6. A composition of claim 5, wherein the epidermal growth factor receptor tyrosine kinase inhibitor is AG1478.

7. A composition of matter comprising at least one miR and at least one additional composition, wherein the miR is upregulated in epidermal growth factor receptor (EGRF) mutant never-smoker cancer cells compared to wild-type never-smoker cancer cells, and wherein the at least one additional composition is useful to treat cancer.

8. A composition of claim 7, wherein the miR is selected from the group: miR-486; miR-126; miR-138; miR-521; miR-451; miR-141; miR-30d; and miR-30a.

9. A composition of matter comprising at least one anti-sense miR and at least one composition, wherein the anti-sense miR is anti-sense to a miR that is upregulated in epidermal growth factor receptor (EGFR) mutant never-smoker cancer cells compared to wild-type never-smoker cancer cells, and wherein the at least one additional composition is useful to treat cancer.

10. A composition of claim 9, wherein the anti-sense miR is selected from a miR that is anti-sense to a miR selected from the group comprising: miR-21; miR-210; and miR-129.

11. A method to identify epidermal growth factor receptor (EGFR) mutant cancer cells in a test sample, comprising comparing miR levels in a test sample to miR levels of a control, wherein differentially-expressed miR levels identify the test sample as containing epidermal growth factor receptor mutant cancer cells.

12. A method of claim 11, wherein the miR are selected from the group comprising: miR-21; miR-210; miR-129; miR-486; miR-126; miR-138; miR-521; miR-451; miR-141; miR-30d; and miR-30a.

13. A method of diagnosing whether a never-smoker subject has, or is at risk for developing, lung cancer, comprising comparing miR levels in a test sample to miR levels of a control, wherein differentially-expressed miR levels diagnoses the subject as either having, or being at risk for developing, lung cancer.

14. A method of claim 13, which further comprises comparing epidermal growth factor receptor mutant status in the test sample and control.

15. A method of claim 14, wherein the epidermal growth factor receptor mutant status is determined using an epidermal growth factor receptor tyrosine kinase inhibitor.

16. A method of claim 13, wherein the miR is selected from the group comprising: miR-21; miR-210; miR-129; miR-486; miR-126; miR-138; miR-521; miR-451; miR-141; miR-30d; and miR-30a.

17. A method to provide a prognosis in a never-smoker cancer patient, comprising: comprising comparing miR levels in a test sample to miR levels of a control, wherein differentially-expressed miR levels indicates a poor prognosis.

18. A method of claim 17, wherein the miR is selected from the group comprising: miR-21; miR-210; miR-129; miR-486; miR-126; miR-138; miR-521; miR-451; miR-141; miR-30d; and miR-30a.

19. A method of diagnosing epidermal growth factor receptor (EGFR) mutant cancer in a patient, comprising comparing miR levels in a test sample to miR levels of a control, wherein differentially-expressed miR levels diagnoses the subject as having epidermal growth factor receptor-mutant cancer.

20. A method of claim 19, wherein the miR is selected from the group comprising: miR-21; miR-210; miR-129; miR-486; miR-126; miR-138; miR-521; miR-451; miR-141; miR-30d; and miR-30a.

21. A method to provide a prognosis in epidermal growth factor receptor (EGFR) mutant cancer patient, comprising: comprising comparing miR levels in a test sample to miR levels of a control, wherein differentially-expressed miR levels indicates a poor prognosis.

22. A method of claim 21, wherein the miR is selected from the group comprising: miR-21; miR-210; miR-129; miR-486; miR-126; miR-138; miR-521; miR-451; miR-141; miR-30d; and miR-30a.

23. A method to treat cancer in a never-smoker patient in need of such treatment, comprising administering a pharmaceutically-effective amount of a composition of claim 1.

24. A method of claim 23, wherein the cancer treated is selected from the group comprising: neuroblastoma; lung cancer; bile duct cancer; non small cell lung carcinoma; hepatocellular carcinoma; lymphoma; nasopharyngeal carcinoma; ovarian cancer; head and neck squamous cell carcinoma; squamous cell cervical carcinoma; gastric cancer; colon cancer; uterine cervical carcinoma; gall bladder cancer; prostate cancer; breast cancer; testicular germ cell tumors; large cell lymphoma; follicular lymphoma; colorectal cancer; malignant pleural mesothelioma; glioma; thyroid cancer; basal cell carcinoma; T cell lymphoma; t(8;17)-prolyphocytic leukemia; myelodysplastic syndrome; pancreatic cancer; t(5;14)(q35.1;q32.2) leukemia; malignant fibrous histiocytoma; gastrointestinal stromal tumor; and hepatoblastoma.

25. A method to treat cancer in a never-smoker patient in need of such treatment, comprising administering a pharmaceutically-effective amount of a composition of claim 4.

26. A method of claim 25, wherein the cancer treated is lung cancer.

27. A method to treat cancer in patient in need of such treatment, comprising administering a pharmaceutically effective amount of a composition of claim 5.

28. A method of claim 27, wherein the cancer treated is adenocarcinoma.

29. A method to treat cancer in a never-smoker patient in need of such treatment, comprising administering a pharmaceutically-effective amount of an anti-sense miR, wherein the antisense miR is antisense to a miR selected from the group comprising: miR-21; miR -210; miR-129.

30. A method to treat cancer in a never-smoker patient in need of such treatment, comprising administering a pharmaceutically-effective amount of an anti-sense miR, wherein the antisense miR is antisense to miR-21.

31. A method of claim 30, wherein the cancer treated is lung cancer.

32. A method of claim 30, wherein the cancer treated is adenocarcinoma.

33. A method of claim 30, which further comprises administering an adjuvant.

34. A method of claim 30, which further comprises administering a compound selected from the group comprising at least one compound selected from the group comprising: a chemotherapy drug; AG1478; gefitinib (Iressa®); erlotinib (Tarceva®); cetuximab; panitumab; zalutumamab; nimotuzamab; matuzumab; and lapatinib.

35. A method of claim 30, which further comprises administering an epidermal growth factor receptor tyrosine kinase inhibitor.

36. A method of claim 30, which further comprises administering AG1478, or a pharmaceutically-acceptable formulation thereof.

37. A method to treat an epidermal growth factor receptor mutant cancer in a patient in need of such treatment, comprising administering a pharmaceutically-effective amount of a composition of claim 1.

38. A method to treat an epidermal growth factor receptor mutant cancer in a patient in need of such treatment, comprising administering a pharmaceutically-effective amount of a composition of claim 4.

39. A method to treat an epidermal growth factor receptor mutant cancer in a patient in need of such treatment, comprising administering a pharmaceutically-effective amount of a miR expression inhibitor, wherein the miR is selected from the group comprising: miR-21; miR-210; and miR-129.

40. A method to treat an epidermal growth factor receptor mutant cancer in a patient in need of such treatment, comprising administering a pharmaceutically-effective amount of a miR-21 expression inhibitor.

41. A method of claim 40, which further comprises administering a compound selected from the group comprising: a chemotherapy drug; AG1478; gefitinib (Iressa®); erlotinib (Tarceva®); cetuximab; panitumab; zalutumamab; nimotuzamab; matuzumab; and lapatinib.

42. A method of claim 40, which further comprises administering an epidermal growth factor receptor tyrosine kinase inhibitor.

43. A method of claim 40, which further comprises administering AG1478, or a pharmaceutically-acceptable formulation thereof.

44. A method to treat an epidermal growth factor receptor mutant cancer in a patient in need of such treatment, comprising administering a pharmaceutically-effective amount of a miR expression promoting composition, wherein the miR is selected from the group comprising: miR-486; miR-126; miR-138; miR-521; miR-451; miR-141; miR-30d; and miR-30a.

45. A method for inducing apoptosis of epidermal growth factor receptor mutant cancer cells, comprising introducing an apoptosis-effective amount of a composition of claim 1.

46. A method for inducing apoptosis of epidermal growth factor receptor mutant cancer cells, comprising introducing an apoptosis-effective amount of a composition of claim 4.

47. A method for inducing apoptosis of epidermal growth factor receptor mutant cancer cells, comprising introducing an apoptosis-effective amount of an anti-sense miR, wherein the antisense miR is antisense to miR-21.

48. A method of claim 47, wherein the epidermal growth factor receptor mutant cancer cells are adenocarcinoma cells.

49. A method of claim 47, wherein adenocarcinoma cells are selected from the group comprising: H3255 cells; H1975 cells; and H1650 cells.

50. A method of claim 47, which further comprises introducing an adjuvant.

51. A method of claim 47, which further comprises introducing a compound selected from the group comprising: a chemotherapy drug; AG1478; gefitinib (Iressa®); erlotinib (Tarceva®); cetuximab; panitumab; zalutumamab; nimotuzamab; matuzumab; and lapatinib.

52. A method of claim 47, which further comprises administering an epidermal growth factor receptor tyrosine kinase inhibitor.

53. A method of claim 47, which further comprises administering AG1478, or a pharmaceutically-acceptable formulation thereof.

54. A method for inducing apoptosis of epidermal growth factor receptor mutant cancer cells, comprising introducing an apoptosis-effective amount of a miR expression inhibitor, wherein the miR is selected from the group comprising: miR-21; miR-210; and miR-129.

55. A method for inducing apoptosis of epidermal growth factor receptor mutant cancer cells, comprising introducing an apoptosis-effective amount of a miR-21 expression inhibitor.

56. A method of claim 54, which further comprises administering a compound selected from the group comprising: a chemotherapy drug; AG1478; gefitinib (Iressa®); erlotinib (Tarceva®); cetuximab; panitumab; zalutumamab; nimotuzamab; matuzumab; and lapatinib.

57. A method of claim 54, which further comprises administering an epidermal growth factor receptor tyrosine kinase inhibitor.

58. A method of claim 54, which further comprises administering AG1478, or a pharmaceutically-acceptable formulation thereof.

59. A method for inducing apoptosis of epidermal growth factor receptor mutant cancer cells, comprising introducing an apoptosis-effective amount of a miR expression promoting composition, wherein the miR is selected from the group comprising: miR-486; miR-126; miR-138; miR-521; miR-451; miR-141; miR-30d; and miR-30a.

60. A method for identifying pharmaceutically-useful compositions, comprising:
i) introducing an anti-sense miR to an epidermal growth factor receptor mutant cancer cell culture, wherein the anti-sense miR is anti-sense to a miR selected from the group comprising: miR-21; miR-210; miR-129;
ii) introducing a test composition to the epidermal growth factor receptor mutant cancer cell culture; and
iii) identifying test compositions which induce apoptosis as pharmaceutically-useful compositions.

61. A method for identifying pharmaceutically-useful compositions, comprising:
i) introducing an anti-sense miR to an epidermal growth factor receptor mutant cancer cell culture, wherein the anti-sense miR is anti-sense to miR-21;
ii) introducing a test composition to the epidermal growth factor receptor mutant cancer cell culture; and
iii) identifying test compositions which induce apoptosis as pharmaceutically-useful compositions.

62. A method of claim 61, wherein the cancer cells are a lung cancer cells.

63. A method of claim 61, which further comprises a step of identifying phosphorylated epidermal growth factor receptor levels.

64. A method of predicting the clinical outcome of a patient diagnosed with lung cancer, comprising detecting the expression level of miR-21 in a cancer cell sample obtained from the patient, wherein a 1.5-fold or greater increase in the level of miR-21 relative to a control, in combination with a epidermal growth factor receptor mutant status predicts a decrease in survival.

65. A method to identify a therapeutic agent for the treatment of lung cancer, comprising screening candidate agents in vitro to select an agent that decreases expression of miR21, thereby identifying an agent for the treatment of lung cancer.

66. A kit for identifying a differentially-expressed miR in lung cancer, comprising at least one molecular identifier of a miR selected from the group comprising: miR-21; miR-210; miR-129; miR-486; miR-126; miR-138; miR-521; miR-451; miR-141; miR-30d; and miR-30a.

67. A kit for identifying a differentially-expressed miR-21 in lung cancer, comprising at least one molecular identifier of miR-21, wherein the molecular identifier is selected from the group comprising: probes; primers; antibodies; or small molecule.
